SEQUENCE LISTING (23 sequences)

SEQ ID NO: 1 - protein, 6 aa, synthetic construct
SSAWIW

SEQ ID NO: 2 - protein, 18 aa, synthetic construct
WIYTGGAGRLYYASWAKG

SEQ ID NO: 3 - protein, 4 aa, synthetic construct
NSAL

SEQ ID NO: 4 - protein, 13 aa, synthetic construct
QSGQSVYNTNQFS

SEQ ID NO: 5 - protein, 7 aa, synthetic construct
DASKLAS

SEQ ID NO: 6 - protein, 13 aa, synthetic construct
LGGYDCSSADCSA

SEQ ID NO: 7 - protein, 132 aa, synthetic construct
METGLRWLLLVAVLKGVQCQSLEESGGDLVKPGASLTLTCKASGFSFGSSAWIWWVRQAPGKGLEGVAWIYTGGAGRLYYASWAKGRFTISKTSSTTVTLQMTSLTAADTATYFCARNSALWGPGTLVTVSS

SEQ ID NO: 8 - protein, 135 aa, synthetic construct
MDTRAPAQLLGLLLLWLPGATFAQVLTQTPSSVSAAVGGTVTINCQSGQSVYNTNQFSWIQQKAGQPPKFLIADASKLASGVPSRFSGSGSGTHFTLTISDVQCDDAATYYCLGGYDCSSADCSAFGGGTEVVVV

SEQ ID NO: 9 - DNA, 396 bp, synthetic construct
  source 1..396: mol_type = other DNA
atggagactgggctgcgctggcttctcctggtcgctgtgctcaaaggtgtccagtgtcagtcgttggaggagtccgggggagacctggtcaagcctggggcatccctgacactcacctgcaaagcctctggattctccttcggtagtagtgcttggatatggtgggtccgccaggctccagggaaggggctggagggggtcgcatggatttatactggtggtgctggtagactttactacgcgagctgggcgaaaggccgattcaccatctccaaaacctcgtcgaccacggtgactctgcaaatgaccagtctgacagccgcggacacggccacctatttctgtgcgagaaattctgcattgtggggcccaggcaccctggtcaccgtctcctca

SEQ ID NO: 10 - DNA, 405 bp, synthetic construct
  source 1..405: mol_type = other DNA
atggacacgagggcacccgctcagctgctggggctcctgctgctctggctcccaggtgccacatttgcccaagtgctgacccagactccatcctccgtgtctgcagctgtgggaggcacagtcaccatcaattgccagtccggtcagagtgtttataataccaaccaattctcctggattcagcagaaagcagggcagcctcccaagttcctgatcgccgatgcatccaaactggcatctggggtcccatcgcggttcagcggcagtggatctgggacacacttcactctcaccatcagcgacgtccagtgtgacgatgctgccacttactactgtctaggcggttatgattgtagtagtgctgattgctctgctttcggcggagggaccgaggtggtggtcgtt

SEQ ID NO: 11 - DNA, 1368 bp, synthetic construct
  source 1..1368: mol_type = other DNA
atggagactgggctgcgctggcttctcctggtcgctgtgctcaaaggtgtccagtgtcagtcgttggaggagtccgggggagacctggtcaagcctggggcatccctgacactcacctgcaaagcctctggattctccttcggtagtagtgcttggatatggtgggtccgccaggctccagggaaggggctggagggggtcgcatggatttatactggtggtgctggtagactttactacgcgagctgggcgaaaggccgattcaccatctccaaaacctcgtcgaccacggtgactctgcaaatgaccagtctgacagccgcggacacggccacctatttctgtgcgagaaattctgcattgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 12 - DNA, 720 bp, synthetic construct
  source 1..720: mol_type = other DNA
atggacacgagggcacccgctcagctgctggggctcctgctgctctggctcccaggtgccacatttgcccaagtgctgacccagactccatcctccgtgtctgcagctgtgggaggcacagtcaccatcaattgccagtccggtcagagtgtttataataccaaccaattctcctggattcagcagaaagcagggcagcctcccaagttcctgatcgccgatgcatccaaactggcatctggggtcccatcgcggttcagcggcagtggatctgggacacacttcactctcaccatcagcgacgtccagtgtgacgatgctgccacttactactgtctaggcggttatgattgtagtagtgctgattgctctgctttcggcggagggaccgaggtggtggtcgttggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 13 - protein, 455 aa, synthetic construct
METGLRWLLLVAVLKGVQCQSLEESGGDLVKPGASLTLTCKASGFSFGSSAWIWWVRQAPGKGLEGVAWIYTGGAGRLYYASWAKGRFTISKTSSTTVTLQMTSLTAADTATYFCARNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 14 - protein, 239 aa, synthetic construct
MDTRAPAQLLGLLLLWLPGATFAQVLTQTPSSVSAAVGGTVTINCQSGQSVYNTNQFSWIQQKAGQPPKFLIADASKLASGVPSRFSGSGSGTHFTLTISDVQCDDAATYYCLGGYDCSSADCSAFGGGTEVVVVGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 15 - DNA, 22 bp, synthetic construct
  misc_feature 1..11: note = DNA
  misc_feature 12: note = RNA
  misc_feature 13..22: note = DNA
  source 1..22: mol_type = other DNA
  modified_base 12: mod_base = m6a
bbbbbbbbbbgabbbbbbbbbb

SEQ ID NO: 16 - DNA, 21 bp, synthetic construct
  source 1..21: mol_type = other DNA
bbbbbbbbbbabbbbbbbbbb

SEQ ID NO: 17 - DNA, 21 bp, synthetic construct
  misc_feature 1..10: note = DNA
  misc_feature 11: note = RNA
  misc_feature 12..21: note = DNA
  source 1..21: mol_type = other DNA
  modified_base 11: mod_base = m6a
bbbbbbbbbbabbbbbbbbbb

SEQ ID NO: 18 - DNA, 22 bp, synthetic construct
  misc_feature 1..11: note = DNA
  misc_feature 12: note = RNA
  misc_feature 13..22: note = DNA
  source 1..22: mol_type = other DNA
  modified_base 12: mod_base = m6a
bbbbbbbbbbaabbbbbbbbbb

SEQ ID NO: 19 - DNA, 22 bp, synthetic construct
  misc_feature 1..11: note = DNA
  misc_feature 12: note = RNA
  misc_feature 13..22: note = DNA
  source 1..22: mol_type = other DNA
  modified_base 12: mod_base = m6a
bbbbbbbbbbcabbbbbbbbbb

SEQ ID NO: 20 - DNA, 21 bp, synthetic construct
  misc_feature 1..10: note = DNA
  misc_feature 11: note = RNA
  misc_feature 12..21: note = DNA
  source 1..21: mol_type = other DNA
  modified_base 11: mod_base = m1a
bbbbbbbbbbabbbbbbbbbb

SEQ ID NO: 21 - DNA, 21 bp, synthetic construct
  misc_feature 1..10: note = DNA
  misc_feature 11: note = RNA
  misc_feature 12..21: note = DNA
  source 1..21: mol_type = other DNA
  modified_base 11: mod_base = m1g
hhhhhhhhhhghhhhhhhhhh

SEQ ID NO: 22 - DNA, 21 bp, synthetic construct
  misc_feature 1..10: note = DNA
  misc_feature 11: note = RNA
  misc_feature 12..21: note = DNA
  source 1..21: mol_type = other DNA
  modified_base 11: mod_base = m22g
hhhhhhhhhhghhhhhhhhhh

SEQ ID NO: 23 - DNA, 21 bp, synthetic construct
  misc_feature 1..10: note = DNA
  misc_feature 11: note = RNA
  misc_feature 12..21: note = DNA
  source 1..21: mol_type = other DNA
  modified_base 11: mod_base = OTHER; note = N6N6-dimethyladenosine (m66A)
bbbbbbbbbbabbbbbbbbbb

(19) United States
(12) Patent Application Publication
(10) Pub. No.: US 2026/0209388 A1
(43) Pub. Date: Jul. 23, 2026

(54) M6A ANTIBODY

(71) Applicants: ABCAM LIMITED, Cambridge (GB); CORNELL UNIVERSITY, Ithaca, NY (US)

(72) Inventors: Velibor SAVIC, Cambridge (GB); Samie JAFFREY, New York, NY (US); Shino MURAKAMI, New York, NY (US)

(21) Appl. No.: 19/145,126

(22) PCT Filed: Jan. 9, 2024
(86) PCT No.: PCT/EP2024/050395

§ 371 Date: Jul. 1, 2025

(30) Foreign Application Priority Data
Jan. 9, 2023 (GB) 2300254.6

(51) Int. Cl. C07K 16/44; A61K 47/68; A61K 49/00; G01N 33/53

(52) U.S. Cl. CPC C07K 16/44; A61K 47/6835; A61K 49/0002; G01N 33/5308

(57) ABSTRACT

Provided are antibodies that are selective for Guanine-N6-methyladenine (Gm6A) over Adenine-N6-methyladenine (Am6A), and methods of producing and using the described antibodies.

### GOVERNMENT SUPPORT

This invention was made with government support under grant number R35NS111631 awarded by the National Institutes of Health. The government has certain rights in the invention.

### FIELD OF THE INVENTION

The invention relates to antibodies that may be used to detect and study methylation of nucleic acids and methods of making and using the same. In particular, provided are antibodies and methods for the detection or quantification of N6-methyladenine (m6A). The provided antibodies and methods may be used to study molecular or physiological processes that affect, or are impacted by, m6A levels.

### BACKGROUND

The modification of DNA and RNA is a well-established phenomenon in biology. There are many known modifications, such as methylation, that are reversibly made by enzymes to DNA and various RNA species. The functional role of these modifications, which may be stably or dynamically made to DNA and/or RNA species, continues to be studied. Improved tools are needed to enable study of DNA and RNA, in particular, RNA methylation.

The methylation of adenine to produce N6-methyladenine (m6A) is a highly prevalent, abundant and conserved internal cotranscriptional modification in eukaryotic RNAs, especially within higher eukaryotic cells. m6A modification has been identified in all major types of RNA species, such as messenger RNAs (mRNAs), transfer RNAs (tRNAs), ribosomal RNAs (rRNAs), circular RNAs (circRNAs), micro RNAs (miRNA), and long non-coding RNAs (lncRNAs). Single-nucleotide resolution m6A mapping has characterized RNA sequences where m6A modification occurs. m6A is highly enriched on mRNA in GGAC motifs as GGm6AC (Linder et al., 2015; Shu et al., 2020; Wang et al., 2020). In 28S and 18S rRNA, m6A is enriched in the AAC motif as Am6AC (Piekna-Przybylska et al., 2008).

m6A modification in mRNA is a dynamic mark. “Writers” (methyltransferases) are responsible for methylation to generate m6A, “erasers” (demethylases) are responsible for demethylation of m6A, and “readers” bind m6A and influence or participate in biological processes.

The “writer complex” is known to contain the methyltransferases METTL3 and METTL14, the METTL3 adapter WTAP4, and other associated proteins including KIAA1429, RBM15/15B, and ZC3H13. Both METTL3 and METTL14 are known to play essential roles in diverse biological processes including embryonic development and neurogenesis. METTL3 has been also shown to regulate cell reprogramming, spermatogenesis, T cell homeostasis, and endothelial-to-hematopoietic transition. METTL14 has been reported to act as a tumor suppressor in glioblastoma and play an oncogenic role in the development and maintenance of acute myeloid leukemia.

The removal/demethylation of m6A is known to be carried out by two different enzymes (erasers): FTO and ALKBH5.

m6A exerts its effects on RNA by recruiting m6A-binding proteins (readers). Knowledge of m6A readers and their functions is rapidly increasing. More and more studies are revealing new m6A readers with specific roles for m6A in different cell types and model systems. Apart from being involved in RNA metabolism, m6A readers also participate in different biological processes, including tumorigenesis, hematopoiesis, virus replication, immune response, and adipogenesis.

m6A has complex functions and has been shown to have key roles in RNA splicing, translation, and stability, as well as the epigenetic effects of certain non-coding RNAs. It is established that regulators of m6A RNA methylation (writers and erasers), as well as readers, are involved in a variety of diseases including human cancers, heart failure, nonalcoholic fatty liver disease and azoospermia (Jiang et al Signal Transduct Target Ther. 2021; 6: 74.). Factors regulating m6A RNA methylation have been shown to have key roles in acute myelocytic leukemia (AML), brain tumors, in particular gliomas, reproductive system related cancers, in particular bladder cancer, and other human diseases including psychiatric disorders, metabolic syndrome, and cardiovascular diseases (Jiang et al Signal Transduct Target Ther. 2021; 6: 74.).

Many methods of detecting, quantifying and functionally studying m6A rely on using m6A antibodies. Various methods have also been developed using antibodies for mapping m6A, based on a combination of immunoprecipitation and sequencing to map m6A throughout the transcriptome of model systems and cell types. However, antibodies directed towards m6A do not selectively distinguish between different RNA species containing the m6A modification. Existing m6A antibodies do not demonstrate selectivity for Gm6A which is enriched in mRNA. Therefore, it can be very difficult to specifically detect Gm6A, or Gm6A-enriched mRNA, for example through immunoprecipitation and subsequent sequencing methods, due to contamination from other RNA species in the sample. Ribosomal RNA (rRNA) is the most abundant class of RNA in the cell. In eukaryotes, rRNA accounts for approximately 80% of total RNA. Among the four eukaryotic rRNAs, 28S and 18S contain one N6-methyladenine (m6A) at A4220 and A1832, respectively (Maden, 1986, 1988). The methylation of S28 is catalyzed by ZCCHC4 (Ma et al., 2019), while 18S is methylated by METTL5 (van Tran et al., 2019).

When attempting to measure m6A modification in mRNA, in order to avoid rRNA contamination, typically one round, or perhaps two rounds, of mRNA isolation is performed to prepare input RNA for bulk m6A measurements. However, it is extremely difficult to completely remove rRNA due to its abundance. Legrand et al. carefully documented the extent of rRNA contamination after mRNA isolation. In this study, the authors show that as much as five rounds of mRNA purification and/or rRNA depletion is required to sufficiently remove rRNA contamination and that the contaminant rRNA affects the quantification of m6A (FIG. 4 in (Legrand et al., 2017)).

ELISA is a particularly attractive method for bulk measurement of m6A levels that can be used in widespread settings. Unlike LC-MS, which requires a LC-MS instrument setting that is highly specialized for nucleotide detection, ELISA is easy to set up in standard laboratory settings. In addition, ELISA can be optimized for a highly standardized and quick protocol, requires low material, and can be performed in a relatively high-throughput manner. Thus, ELISA is employed in numerous studies to quantify m6A levels in various biological samples (Bornaque et al., 2022; Chang et al., 2022; Wu et al., 2019; Yin et al., 2022). However, RNA samples used in these studies for m6A quantification may suffer from the contamination of rRNA. In case of bulk m6A measurement by methods such as ELISA and LC-MS, the total m6A levels in the sample will be measured as a bulk, and the origin of m6A is indistinguishable. Thus, any contamination of rRNA can be deleterious for accurate quantification of m6A from mRNA.

The contamination by rRNA is also evident when m6A is mapped across transcriptome. Methylated RNA immunoprecipitation sequencing (MeRIP-seq) and m6A individual-nucleotide-resolution cross-linking and immunoprecipitation (miCLIP) are the most commonly used methods to map m6A across the transcriptome. miCLIP was first described in Linder et al. 2015 using the Abcam m6A antibody ab151230. In these methods, mRNA is isolated using oligo(dT) beads, fragmented, and immunoprecipitated using m6A antibodies. After extensive washing, the immunoprecipitated RNA is converted to sequencing libraries and subjected to sequencing. Unlike the bulk measurement by ELISA or LS-MS, m6A mapping does identify the origin of m6A by mapping the sequencing data to the reference genome. Despite the mRNA isolation step, immunoprecipitation of m6A using m6A antibodies currently on the market capture significant m6A from rRNAs arising from rRNA contamination. In a highly cited review by Patil et al, the authors show highly enriched m6A signal from rRNA in miCLIP libraries (FIG. 5 in (Patil et al., 2018)). The high read coverage over rRNAs compromises the coverage of m6A mapping over mRNAs, thus rRNA contamination is a significant problem in m6A mapping. Therefore, sequencing m6A in specific RNA species such as mRNA based on immunoprecipitation with m6A antibodies is challenging.

Thus, it is a major challenge in the field to develop more targeted tools and methods that quantify m6A in specific RNA species, in particular in mRNA but not rRNA. In addition, there is a need for antibodies that target m6A on mRNA and allow direct visualization of the m6A-modified mRNA.

### SUMMARY OF INVENTION

The present invention provides antibodies that bind to targets comprising Guanine-N6-methyladenine (Gm6A), particularly RNA or DNA comprising Gm6A. Provided are antibodies that are selective for Guanine-N6-methyladenine (Gm6A) over Adenine-N6-methyladenine (Am6A), and preferably selectively bind targets comprising m6A in a GAC context (Gm6AC) compared to an AAC context (Am6AC). Also provided are methods of producing and/or using the described antibodies. The present invention provides methods of binding, detecting and/or monitoring Gm6A. The present invention further provides means and methods to diagnose, prevent, alleviate and/or treat a disease, a disorder and/or abnormality associated with aberrant Gm6A.

Aspects and embodiments of the invention are set out in the claims and in the detailed description.

### DETAILED DESCRIPTION OF EMBODIMENTS OF THE INVENTION

The present inventors have generated antibodies which bind to targets comprising Guanine-N6-methyladenine (Gm6A). As mentioned in the Examples, antibodies were raised against the Gm6A-comprising compound disclosed in FIG. 1. Several antibodies that bind oligonucleotides comprising m6A were generated, but only one of these antibodies, denoted herein 21H1L4, was selective for Gm6A over other methylated nucleotides such as Adenine-N6-methyladenine (Am6A). Antibody 21H1L4 is characterized herein and represents an example of the antibodies provided herein.

A class of antibodies is provided herein. In some instances, the properties of the provided antibodies are discussed herein using the singular merely to make it clear that the discussed properties are all possessed by the same antibody, rather than being the combined properties of a mixture of distinct antibodies. It should nevertheless be understood that the properties discussed herein are those of the provided class of antibodies. Any reference herein to “an antibody”, “a provided antibody”, “the antibody”, “the provided antibody”, “an antibody provided herein” or “the antibody provided herein” and variations thereof should be understood to encompass any one of the antibodies of the provided class of antibodies, unless explicitly stated otherwise.

Using oligonucleotides comprising Gm6A and oligonucleotides lacking Gm6A and instead comprising alternative methylated nucleotides, the inventors have demonstrated selectivity of the exemplified antibody (21H1L4) for Gm6A over other methylated nucleotides such as Adenine-N6-methyladenine (Am6A).

Thus, in one aspect, provided are antibodies selective for Guanine-N6-methyladenine (Gm6A) over Adenine-N6-methyladenine (Am6A). The term “selective” means that the antibody exhibits significantly more binding to the target than to the relevant comparator. Thus, the provided antibody exhibits significantly more binding to Gm6A (or a target comprising Gm6A that is free from A6 mA, which may, for example, be an oligonucleotide comprising or consisting of SEQ ID NO: 15) than to A6 mA (or a Gm6A-free target comprising Am6A, which may, for example, be an oligonucleotide comprising or consisting of SEQ ID NO: 18). The selectivity, i.e. difference in binding to the target versus the comparator, is preferably at least 2-fold, at least 4-fold, at least 6-fold, at least 8-fold, at least 10-fold, at least 20-fold, at least 40-fold, at least 50-fold, at least 80-fold, at least 100-fold or at least 200-fold. Preferably, the antibody exhibits no significant binding to Am6A, no visually detectable binding to Am6A, or no detectable binding to Am6A.

The provided antibodies may be further selective for Gm6A over Cytosine-N6-methyladenine (Cm6A). They may further be selective for Gm6A over Gm6A over N6-methyladenine (m6A), N6 N6 dimethyladenine (m6, 6A), N1-methyladenine (m1A), N1-methylguanine (m1G), and/or N2,N2-dimethylguanine (m22G). They may be selective for GGm6AC over Am6AC.

Antibody selectivity for Gm6A over a comparator modification, such as Am6A, may be determined by assaying antibody binding to (i) a target, such as an oligonucleotide, that comprises Gm6A; and (ii) a Gm6A-free comparator, for example a Gm6A-free oligonucleotide, that comprises a comparator modification, such as Am6A. The level of antibody binding to the target may be compared to the level of antibody binding to the comparator. The comparison may be qualitative, such as a visual assessment of the presence or absence of a visual signal; semi-quantitative, such as a visual assessment of the intensity of a visual signal; or quantitative, such as measurement of the intensity of a signal. Suitable methods for assaying antibody binding include dot blot; homogeneous time-resolved fluorescence (HTRF); and/or immunocytochemistry (ICC) experiments. Thus, the antibody selectivity may be as determined using one or more of dot blot; homogeneous time-resolved fluorescence (HTRF); and/or immunocytochemistry (ICC) experiments, preferably in accordance with the methods described in the Examples herein.

The inventors have demonstrated binding of the exemplified antibody (21H1L4) to synthetic oligonucleotides comprising Gm6A and to total RNA of wild-type cells, whereas the antibody exhibits significantly less or no detectable binding to comparator synthetic oligonucleotides lacking Gm6A and to total RNA of Mettl3 (a Gm6A methylase) knock out cells.

Accordingly, the antibodies provided herein bind a target comprising Guanine-N6-methyladenine (Gm6A). The binding is preferably specific. By “specific” binding is meant that the antibody binds to a target comprising Gm6A with significant affinity and does not bind, or binds with significantly lower affinity, to one or more controls lacking Gm6A. In particular, the antibody binds to a nucleic acid molecule comprising Gm6A and does not bind, or does not significantly bind, to a corresponding nucleic acid molecule lacking Gm6A. Controls may also include other cellular components such as proteins, lipids and carbohydrates.

The difference in binding to the target versus the control is preferably at least is preferably at least 2-fold, at least 4-fold, at least 6-fold, at least 8-fold, at least 10-fold, at least 20-fold, at least 40-fold, at least 50-fold, at least 80-fold, at least 100-fold or at least 200-fold. Preferably, the antibody exhibits no significant binding to a control, no visually detectable binding to control, or no detectable binding to control.

Antibody specificity may be determined using, for example, one or more of dot blot; homogeneous time-resolved fluorescence (HTRF); and/or immunocytochemistry (ICC) experiments, preferably in accordance with the methods described in the Examples herein.

Shepherd and Dean (2000), Monoclonal Antibodies: A Practical Approach, Oxford University Press and/or Howard and Bethell, provides a description of immunoassay formats and conditions that can be used to determine specific immunoreactivity.

An assay for antibody specificity may include a step of preincubating the antibody with (i) a target comprising Gm6A; or (ii) a control lacking Gm6A, under conditions that allow binding of the antibody to its target, prior to assaying target binding. Preincubation with the target should fill the antibody binding sites and significantly reduce target binding in the assay, whereas preincubation with a control should not have a significant impact on target binding.

“N6-methyladenine” or “m6A” or “m⁶A”, which are used synonymously herein, denote adenine that has been methylated, and the chemical formula thereof is shown in FIG. 8. In the art, the term “m6A” is sometimes used colloquially to refer to N6-methyladenosine and/or N6-methyldeoxyadenosine, because m6A is typically present in a nucleic acid molecule. A more precise terminology is used herein unless stated otherwise, whereby “m6A” denotes the N6-methyladenine moiety, which may, of course, be linked to another moiety, for example be part of a larger molecule. Thus, the m6A may be in the form of, for example, N6-methyladenosine or N6-methyldeoxyadenosine. In some embodiments, N6-methyladenine is in the form of N6-methyladenosine and is preferably part of an RNA molecule. In other embodiments, N6-methyladenine is in the form of N6-methyldeoxyadenosine and is preferably part of a DNA molecule. The terms “Gm6A” or “Guanine-N6-methyladenine” or “G-m6A”, which are used synonymously herein, denote a motif comprising guanine and m6A, wherein the spatial arrangement of guanine relative to m6A is as it is within a nucleic acid molecule that comprises m6A preceded by a guanine, for example as a result of METTL3 methylation. Thus, the motif preferably comprises guanine linked to m6A via a sugar backbone, for example a ribose or deoxyribose backbone that preferably comprises a phosphodiester bond. Thus, in Gm6A the m6A is preferably the form of N6-methyladenosine or N6-methyldeoxyadenosine and the G is preferably in the form of guanosine or deoxyguanosine. In some embodiments, Gm6A is in the form of guanosine linked to N6-methyladenosine via a phosphodiester bond between the ribose moieties and is preferably part of an RNA molecule. In other embodiments, Gm6A is in the form of deoxyguanosine linked to N6-methyldeoxyadenosine via a phosphodiester bond between the deoxyribose moieties and is preferably part of a DNA molecule.

It will be understood that these features apply mutatis mutandis where further nucleobases are mentioned, for example “Gm6AC”, “GGm6AC” and “Am6AC” all denote motifs to which these features apply mutatis mutandis. Thus, for example “Gm6AC” denotes a motif that may be formed by guanosine linked to N6-methyladenosine which in turn is linked to cytosine; or deoxyguanosine linked to N6-methyldeoxyadenosine which in turn is linked to deoxycytosine; wherein each link is via a phosphodiester bond between the respective sugar moieties.

Any reference herein to a “phosphodiester bond” is to the type of bond naturally found in nucleic acid molecules such as RNA or DNA.

Gm6A may be within an RNA molecule or a DNA molecule, preferably Gm6A is within an RNA molecule such as an mRNA molecule.

The pattern of binding of the exemplified antibody 21H1L4 to a nucleic acid molecule comprising Gm6A, but not (or not significantly) to a corresponding nucleic acid molecule lacking Gm6A provides insights into its epitope. Accordingly, the antibodies provided herein may bind an epitope comprising or consisting of Gm6A, wherein the Gm6A may be in any of the forms described above, preferably guanosine linked to N6-methyladenosine via a phosphodiester bond between the ribose moieties, or deoxyguanosine linked to N6-methyldeoxyadenosine via a phosphodiester bond between the deoxyribose moieties. The Gm6A modification is typically found in mRNA and is typically preceded by a G. Accordingly, the antibodies provided herein preferably bind an epitope comprising, consisting essentially or, or consisting of GGm6A or Gm6A, preferably Gm6A. Preferably, the epitope is located within a nucleic acid molecule. Details of nucleic acid molecules are provided elsewhere herein.

Methods for testing the binding of an antibody to a target epitope are known. Methods for identifying the epitope that an antibody binds to are also known and may, for example, include target binding followed by crystallization.

The specific antibody 21H1L4, which is disclosed herein in detail, can bind to Gm6A (or a target comprising Gm6A). The provided antibodies may be defined by their ability to compete for binding to Gm6A (or a target comprising Gm6A) with antibody 21H1L4. Thus, provided are antibodies that are selective for Gm6A over Am6A and can compete for binding to Gm6A (or a target comprising Gm6A) with another antibody that is selective for Gm6A over Am6A (a “reference antibody”), such as antibody 21H1L4.

Such “competing” antibodies may bind to the same epitope as antibody 21H1L4, substantially the same epitope as antibody 21H1L4, or to an epitope that overlaps with the epitope of antibody 21H1L4 (but must be selective for Gm6A over Am6A).

The “competing” antibodies should be functionally equivalent to antibody 21H1L4 at least in respect of their ability to be selective for Gm6A over Am6A. They may further be functionally equivalent to antibody 21H1L4 in respect of one or more further characteristics disclosed herein, for example, selectivity for Gm6A over Cm6A, selectivity for Gm6A over one or more of N6-methyladenine (m6A), N6 N6 dimethyladenine (m6, 6A), N1-methylguanine (m1G), and/or N2,N2-dimethylguanine (m22G), and/or selectivity for Gm6A over m1A.

By “functionally equivalent” is meant that the identified functional characteristic(s) is/are shared by both molecules. Where the functional characteristic is quantifiable, the functional equivalent should vary from the reference molecule by no more than 50, 40, or 30%, preferably no more than 20, 15, 10, or 5% when tested under the same conditions. Alternatively, the functional equivalent may vary from the reference molecule by no more than 2-fold, 4-fold, 6-fold, 8-fold or 10-fold when tested under the same conditions. Thus, an antibody that is “functionally equivalent” to antibody 21H1L4 at least in respect of their ability to be selective for Gm6A over Am6A should have a selectivity for Gm6A over Am6A that is within 50, 40, 30, 20% or less of the selectivity displayed by antibody 21H1L4 when tested under the same conditions.

Where within an antibody a variant CDR sequence is “functionally equivalent” to a reference CDR sequence that it is substantially homologous to, the antibody with the variant CDR sequence must retain its functional characteristics, but, for example, the affinity for the target antigen may be altered, provided that it differs from the affinity of the reference antibody by no more than 50, 40, or 30%, preferably no more than 20, 15, 10, or 5% when tested under the same conditions.

The “competing” antibodies may share structural similarity with antibody 21H1L4. For example, they may have one or more of the sequences disclosed herein. In particular, the “competing” antibodies may have one or more sequences that are substantially homologous to the SEQ ID Nos disclosed herein, as discussed below.

The term “antibody” refers to all isotypes of immunoglobulins (IgG, IgA, IgE, IgM, IgD, and IgY) including various monomeric, polymeric and chimeric forms, unless otherwise specified. Specifically encompassed by the term “antibody” are isolated antibodies, monoclonal antibodies (mAbs), recombinant antibodies, chimeric antibodies and humanized antibodies. Preferably, the antibody is a monoclonal antibody. The term “isolated” refers to antibodies (or nucleic acids) that are distinguished from any such components that may be present in situ within a human or animal body or a tissue sample derived from a human or animal body. The sequences may, however, correspond to or be substantially homologous to sequences as found in a human or animal body. The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The term “recombinant antibody” includes all antibodies that are prepared, expressed, created or isolated by recombinant means, such as antibodies isolated from an animal (e.g., a rabbit) that is transgenic for human immunoglobulin genes; antibodies expressed using a recombinant expression vector transfected into a host cell, antibodies isolated from a recombinant, combinatorial antibody library, or antibodies prepared, expressed, created or isolated by any other means that involves splicing of (e.g. human) immunoglobulin gene sequences to other DNA sequences. The term “chimeric antibodies”, refers to an antibody, which comprises a variable region provided herein fused or chimerized with an antibody region (e.g., constant region) from another species, that may be human or non-human, e.g., mouse, horse, rabbit, dog, cow, or chicken. The term “humanized antibody” refers to “humanized” forms of non-human (e.g. murine or rabbit) antibodies which are chimeric immunoglobulins which contain minimal sequence derived from non-human immunoglobulin. In some embodiments, the antibody may be a “fully-human antibody” which as used herein refers to an antibody which comprises human immunoglobulin protein sequences only. A fully human antibody may contain murine carbohydrate chains if produced in a mouse, in a mouse cell or in a hybridoma derived from a mouse cell.

In some embodiments, the antibody is an IgA, IgD, IgE, IgM, or IgG antibody, preferably an IgG antibody. In some embodiments, the antibody is a bivalent or polyvalent antibody comprising at least two antigen binding fragments of the antibody. Bivalent means comprising two antigen binding sites. Polyvalent as used herein, means comprising two or more antigen binding sites, thus being able to bind two or more antigens, which may have the same or a different structure, simultaneously.

Antibodies have variable heavy chain and variable light chain complementary determining regions (CDRs). The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each of the variable heavy and variable light domains (VH CDR1, VH CDR2, VH CDR3, VL CDR1, CL CDR2 and VL CDR3). The CDR regions of an Ig-derived region may be determined as described in Kabat “Sequences of Proteins of Immunological Interest”, 5th edit. NIH Publication no. 91-3242 U.S. Department of Health and Human Services (1991). CDR sequences provided herein are defined according to Kabat. However, it will be understood by the skilled person that the invention is intended to encompass antibodies in which the CDR sequences are defined according to any useful identification/numbering scheme. For example, Chothia (Canonical structures for the hypervariable regions of immunoglobulins. Chothia C, Lesk A M. J Mol Biol. 1987 Aug. 20; 196(4):901-17), IMGT (IMGT, the international ImMunoGeneTics database. Giudicelli V, Chaume D, Bodmer J, Müller W, Busin C, Marsh S, Bontrop R, Marc L, Malik A, Lefranc M P. Nucleic Acids Res. 1997 Jan. 1; 25(1):206-11 and Unique database numbering system for immunogenetic analysis. Lefranc M P. Immunol Today. 1997 November; 18(11):509), MacCallum (MacCallum R M, Martin A C, Thornton J M, J Mol Biol. 1996 Oct. 11; 262(5):732-45) and Martin (Abhinandan K R, Martin A C R. Analysis and improvements to Kabat and structurally correct numbering of antibody variable domains. Mol Immunol. (2008) 45:3832-9.

The antibodies provided herein comprise a Heavy Chain Variable Region comprising a variable heavy (VH) CDR1, a VH CDR2 and a VH CDR3; and a Light Chain Variable Region comprising a variable light (VL) CDR1, a VL CDR2 and a VL CDR3. The antibodies provided herein may comprise one or more of the specific amino acid sequences disclosed herein with a SEQ ID NO (See Tables 3-4 and 6), or a sequence substantially homologous thereto. For example, the antibodies may comprise 1, 2, 3, 4, 5 or all of the following sequences:
(i) a (VH) CDR1 that has the amino acid sequence of SEQ ID NO: 1 or a sequence substantially homologous thereto;
(ii) a VH CDR2 that has the amino acid sequence of SEQ ID NO: 2 or a sequence substantially homologous thereto;
(iii) a VH CDR3 that has the amino acid sequence of SEQ ID NO: 3 or a sequence substantially homologous thereto;
(iv) a (VL) CDR1 that has the amino acid sequence of SEQ ID NO: 4 or a sequence substantially homologous thereto;
(v) a VL CDR2 that has the amino acid sequence of SEQ ID NO: 5 or a sequence substantially homologous thereto; and/or
(vi) a VL CDR3 that has the amino acid sequence of SEQ ID NO: 6 or a sequence substantially homologous thereto.

For example, the antibodies may comprise
a) a Heavy Chain Variable Region comprising:
(i) a variable heavy (VH) CDR1 that has the amino acid sequence of SEQ ID NO: 1 or a sequence substantially homologous thereto;
(ii) a VH CDR2 that has the amino acid sequence of SEQ ID NO: 2 or a sequence substantially homologous thereto; and
(iii) a VH CDR3 that has the amino acid sequence of SEQ ID NO: 3 or a sequence substantially homologous thereto; and
b) a Light Chain Variable Region comprising:
(iv) a variable light (VL) CDR1 that has the amino acid sequence of SEQ ID NO: 4 or a sequence substantially homologous thereto;
(v) a VL CDR2 that has the amino acid sequence of SEQ ID NO: 5 or a sequence substantially homologous thereto; and
(vi) a VL CDR3 that has the amino acid sequence of SEQ ID NO: 6 or a sequence substantially homologous thereto.

Any reference herein that a region “has” or “having” a particular sequence means that is comprises, consists essentially, or consists of that sequence, preferably consists of it.

The term “substantially homologous” means that a particular subject sequence, for example, a mutant sequence, is structurally similar to its reference sequence but varies from its reference sequence by one or more amino acid (or nucleotide, in the case of a nucleic acid sequence) substitutions, deletions, and/or additions; and is functionally equivalent to the reference sequence. Functional equivalence is defined elsewhere herein.

Each substantially homologous sequence may be a sequence containing exactly or no more than 5, 4, 3, 2 or 1 amino acid substitutions, deletions, and/or additions of the given CDR sequence.

In some embodiments, the “substantially homologous” sequence differs from the reference sequence only through substitutions. In some embodiments, the substitutions include only “exemplary substitutions” (see Table 1) or conservative substitutions. In some embodiments, the substitutions include only conservative substitutions.

In some embodiments, the provided antibody comprises exactly or no more than 20, 19, 18, 17, 16, 15, 14, 13, 12, 11, 10, 9, 8, 7, 6, 5, 4, 3, 2 or 1 amino acid substitutions, deletions, and/or additions in total across all 6 CDRs compared to the recited CDR sequences. In some embodiments, the antibody CDR sequences differ from the reference CDRs only in that the antibody comprises exactly or no more than 18, 15, 12, 10, 8, 6, 4 or 2 substitutions in total across all 6 CDRs compared to the recited CDR sequences, wherein each CDR sequence comprises exactly or no more than 3, 2 or 1 amino acid substitutions per CDR sequence compared to the recited CDR sequences, and wherein optionally all substitutions are conservative substitutions. In some embodiments, the CDR sequences of the provided antibody differ from the reference CDRs only in that the antibody comprises exactly or no more than 10, 9, 8, 7, 6, 5, 4, 3, 2 or 1 amino acid substitutions in total across all 6 CDRs compared to the recited CDR sequences, wherein optionally all substitutions are conservative substitutions.

TABLE 1

| | | Conservative |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gin |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The antibodies provided herein may have Heavy Chain Variable Region and/or Light Heavy Chain Variable Region having an amino acid sequence with a SEQ ID NO disclosed herein, or a sequence having a percentage identify thereto. Thus, the antibodies provided herein may comprise a Heavy Chain Variable Region comprising the sequence of SEQ ID NO: 7 or a Heavy Chain Variable Region having at least 70%, 75%, 80%, 85%, 90% 91%, 92%, 93%, 94%, 95%, 96%, 97%, 98% or 99% identity thereto; and/or comprises a Light Chain Variable Region comprising the sequence of SEQ ID NO: 8 or a Light Chain Variable Region having at least 70%, 75%, 80%, 85%, 90% 91%, 92%, 93%, 94%, 95%, 96%, 97%, 98% or 99% identity thereto.

The antibodies provided herein may have Heavy Chain and/or Light Chain having an amino acid sequence with a SEQ ID NO disclosed herein, or a sequence having a percentage identify thereto. Thus, the antibodies provided herein may comprise a Heavy Chain comprising the sequence of SEQ ID NO: 13 or a Heavy Chain having at least 70%, 75%, 80%, 85%, 90% 91%, 92%, 93%, 94%, 95%, 96%, 97%, 98% or 99% identity thereto; and/or comprises a Light Chain comprising the sequence of SEQ ID NO: 14 or a Light Chain having at least 70%, 75%, 80%, 85%, 90% 91%, 92%, 93%, 94%, 95%, 96%, 97%, 98% or 99% identity thereto.

When a sequence is defined as having a defined sequence identity percentage to a reference molecule, for example an antibody has a Heavy Chain Variable Region having a sequence at least 90% identical to SEQ ID NO: 7, the percentage identity is determined across the full length of the reference molecule (e.g. SEQ ID NO: 7). The percent identity between two sequences is a function of the number of identical positions shared by the sequences (i.e., % identity=number of identical positions/total number of positions×100), taking into account the number of gaps, and the length of each gap, which need to be introduced for optimal alignment of the two sequences.

The term “antibody” is used herein to refer to a protein comprising a Heavy Chain Variable Region and Light Heavy Chain Variable Region that is able to bind specifically to its target antigen. Thus, the term “antibody” encompasses a full-length immunoglobulin (Ig) comprising two heavy chains (each comprising a variable region and constant regions CH1, CH2 and CH3) and two light chains (each comprising a variable region and a constant region CL); as well as antigen-binding fragments. For example, the antibodies may be selected from an IgG or an antigen-binding fragment (of an antibody), such as a Fab, Fab′, F(ab′)2, Fv, scFv, diabody, monovalent IgG, scFv-Fc, minibody, or triabody. The term “antigen-binding fragment of an antibody”, refers to a molecule other than a full-length antibody that comprises a portion of a full-length antibody and that binds the antigen to which the full-length antibody binds. A “Fab” as used herein is comprised of one light chain and the CH1 and variable regions of one heavy chain. The heavy chain of a Fab molecule cannot form a disulfide bond with another heavy chain molecule. A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a “F(ab′) 2” molecule. A “F(ab′)2 fragment” as used herein contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains. The “Fv region” comprises the variable regions from both the heavy and light chains, but lacks the constant regions. “Single-chain Fv” or “scFv” antibody fragments have, in the context of the invention, the VH and VL domains of an antibody, wherein these domains are present in a single polypeptide chain. Generally, the scFv polypeptide further comprises a polypeptide linker between the VH and VL domains which enables the scFv to form the desired structure for antigen binding. The term “diabody” or “diabodies” refers to antibody fragments with two antigen-binding sites, which fragments can comprise a heavy chain variable domain (VH) connected to a light chain variable domain (VL) in the same polypeptide chain.

The antibodies provided herein may comprise a detectable label. Detectable labels that allow the detection of an antibody without the antibody losing its activity are known in the art and may thus for example be selected from fluorescent labels, enzyme labels, biotin, magnetic beads, agarose beads, magnetic agarose beads and colloidal gold.

In another aspect, provided is a nucleic acid molecule encoding one or more of the sequences disclosed herein, for example a VH CDR1, VH CDR2, VH CDR3, VL CDR1, VL CDR2 and/or VL CDR3 disclosed herein. Thus, provided is a nucleic acid molecule comprising a nucleotide sequence region encoding a variable heavy chain region and/or a variable light chain region of an antibody provided herein. The nucleic acid may comprise one or more of the specific nucleic acid sequences disclosed herein with a SEQ ID NO (See Tables 2 and 5), or a sequence substantially homologous thereto. The nucleic acid molecule may comprise a nucleotide sequence region encoding an antibody provided herein, or a plurality (2 or more) nucleotide sequence regions that together encode an antibody provided herein. Alternatively, provided is a plurality of nucleic acid molecules comprising the nucleotide sequence regions that (together) encode an antibody provided herein. The nucleic acid molecule(s) may be used to express the provided antibodies. It or they may be comprised in an expression vector or set of expression vectors. For example, provided is a set of expression vectors comprising a first vector encoding at least the Heavy Chain Variable Region of an antibody provided herein, and optionally also the heavy chain constant regions; and a second vector encoding at least the Light Heavy Chain Variable Region of an antibody provided herein, and optionally also the light chain constant region.

An “expression vector” is a replicon, such as plasmid, phage, cosmid, or virus in a nucleic acid molecule as described above may be operably inserted so as to bring about the expression of the inserted nucleic acid molecule. The nucleic acid molecule(s), expression vector or set of expression vectors may be comprised in a cell-free expression system. Alternatively, a host cell may comprise a nucleic acid molecule provided herein, the set of nucleic acid molecules provided herein, the expression vector provided herein and/or the set of expression vectors provided herein. “Host cells” provided herein, such as host cells for cloning or expression of antibody-encoding vectors, include prokaryotic or eukaryotic cells.

In another aspect, provided are methods of producing the provided antibodies. Appropriate methods known in the art include cell-based production methods and cell-free production methods. For example, provided is a method of producing an antibody, comprising culturing a host cell that comprises the expression vector or set of expression vectors provided herein, under conditions effective to express the encoded antibody. Optionally, the method further comprises isolating the expressed antibody.

In another aspect, provided are methods for producing or generating antibodies that are selective for Gm6A over Am6A. The methods may involve a first step of immunizing cells with an antigen comprising Gm6A to generate antibodies that bind Gm6A. Preferably, the antigen used for immunization comprises or consists of carboxy C2 dG m6A or a Gm6A-comprising variant thereof. Most preferably, the antigen consists of carboxy C2 dG m6A.

Alternatively, the methods may involve a first step of generating antibodies that bind Gm6A by creating a variant of antibody 21H1L4 that differs in one or more amino acids from the sequence of antibody 21H1L4. Variants may be created for example through site-specific or random mutagenesis in one or more CDRs and/or framework regions; or through chain shuffling and the like. The 6 CDR sequences of the variants are preferably substantially homologous to the CDR sequences disclosed herein.

The methods may optionally further involve a step of screening the generated antibodies for their ability to bind Gm6A. The ability of an antibody to bind Gm6A may be determined by testing binding to a target comprising Gm6A versus binding to a target lacking Gm6A, as discussed elsewhere herein.

The methods may further involve a step of screening the generated antibodies for selectivity for Gm6A over Am6A. Such selectivity may be assayed using a suitable test substrate comprising Gm6A and a suitable control substrate lacking Gm6A, but comprising Am6A. Suitable screening methods and test and control substrates are disclosed in the present Examples, such as dot blots (in vitro or ex vivo) or immunocytochemistry. Thus, the test substrate comprising Gm6A may, for example, comprise or consist of SEQ ID NO: 15, or may be designed based on the principles embodied by SEQ ID NO: 15. The control substrate may, for example, comprise or consist of SEQ ID NO: 18, or may be designed based on the principles embodied by SEQ ID NO: 18.

The methods may further comprise the steps of obtaining an amplified antibody gene for the generated antibody by a PCR amplification means and then expressing and preparing the generated antibody or a protein similar to the generated antibody by host cells.

The antibodies provided herein are selective for Gm6A over Am6A. Gm6A is highly abundant on mRNA and Am6A is highly abundant on ribosomal RNA (rRNA), such as 28S and 18S rRNA. It is therefore postulated that the antibodies provided herein are selective for messenger RNA (mRNA) over rRNA, such as 28S and/or 18S rRNA. By contrast, prior art antibodies that bind m6A are not selective for Gm6A over Am6A and consequently exhibit significant binding to rRNA. The provided antibodies may therefore advantageously be used to analyse m6A levels of mRNA.

In a further aspect, provided are conjugates comprising any one of the antibodies provided herein conjugated to an agent. The agent may, for example, be a selectable tag, such as a His tag; a detectable label, details of which are provided elsewhere herein; a toxin; or a therapeutic agent, for example a different antibody, a small chemical molecule, a nucleic acid-based therapeutic and the like. More particularly, the agent may comprise or consist of an enzyme, avidin, streptavidin, biotin, Protein A, Protein G, magnetic beads, fluorophores, metal, or radioactive isotopes such as radioconjugates. The therapeutic agent may be any agent that achieves a therapeutic effect for preventing, alleviating or treating a disease either independently or synergistically with the antibody. Thus, the conjugate may be an immunoconjugate.

In a further aspect, provided are compositions comprising an antibody provided herein (i.e. any one of the antibodies provided herein) or a conjugate provided herein (i.e. any one of the conjugates provided herein). The form of the composition may be appropriately tailored for storage or use of the antibody or conjugate.

In particular, provided are pharmaceutical compositions comprising an antibody provided herein or a conjugate provided herein, and a pharmaceutically acceptable carrier, diluent, excipient, adjuvant and/or excipient. Pharmaceutically acceptable carriers, diluents, adjuvants and excipients are well known in the pharmaceutical art and are described, for example, in Remington's Pharmaceutical Sciences, 15th or 18th Ed. (Alfonso R. Gennaro, ed.; Mack Publishing Company, Easton, PA, 1990); Remington: the Science and Practice of Pharmacy 19th Ed. (Lippincott, Williams & Wilkins, 1995); Handbook of Pharmaceutical Excipients, 3rd Ed. (Arthur H. Kibbe, ed.; Amer. Pharmaceutical Assoc, 1999); Pharmaceutical Codex: Principles and Practice of Pharmaceutics 12th Ed. (Walter Lund ed.; Pharmaceutical Press, London, 1994); The United States Pharmacopeia: The National Formulary (United States Pharmacopeial Convention); Fiedler's “Lexikon der Hilfstoffe” 5th Ed., Edition Cantor Verlag Aulendorf 2002; “The Handbook of Pharmaceutical Excipients”, 4th Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference. The carriers, diluents, adjuvants and pharmaceutical excipients can be selected with regard to the intended route of administration and standard pharmaceutical practice.

The antibodies and conjugates provided herein and may be used to bind, detect, analyze, monitor or quantify Gm6A in a sample or in a subject. Accordingly, in further aspects, provided are uses of the provided antibodies and/or conjugates and related methods for binding, detecting, analyzing, monitoring or quantifying Gm6A. This may allow an analysis of the regulation of m6A modifications, for example the regulation of one or more m6A writers, m6A erasers and/or m6A readers.

The methods and uses may be performed in vitro, ex vivo or in vivo, for example on an in vitro or ex vivo sample.

The “sample” may be any nucleic acid comprising sample. It may be a synthetic sample or a biological sample, such as a biological fluid, cell culture, cell sample, tissue sample, or a combination or two or more of the above. The sample may have been isolated from a subject. A “tissue sample” may comprise, e.g., surgically resected tumor tissue or a biopsy. A “biological fluid” may, for example, comprise or consist of blood or a blood product such as serum or plasma; lymph; saliva; sputum; synovial fluid; peritoneal fluid; breast milk; semen; bronchoalveolar lavage; urine; and/or faeces. The sample may be or prokaryotic or eukaryotic origin. For example, it may have been derived from a subject. Any reference to a “subject” should be understood to be a human or non-human animal, such as livestock or a companion animal, but is preferably a human. The sample should ideally comprise nucleic acids, which may be synthetic, and/or may be from one or more prokaryotic and/or eukaryotic sources. The sample should ideally comprise RNA molecules and preferably comprise at least mRNA. The RNA molecules may be single stranded (ssRNA). The skilled person will understand that by “ssRNA” is meant a single nucleic acid molecule, irrespective of the presence or absence of any internal base-pairing that may create secondary structures.

The sample may have been processed to make the nucleic acid content thereof more accessible, for example through permeabilization or lysis of cell membranes.

Alternatively or in addition, the sample may have been processed to enrich the nucleic acid content thereof, or the content of one or more types of nucleic acids, such as DNA or RNA, preferably RNA. This may involve removing at least a proportion of the protein, lipid and/or carbohydrate components of the sample.

Alternatively or in addition, the sample may have been processed to purify, or at least partially purify, the nucleic acid content thereof, or the content of one or more types of nucleic acids, such as DNA or RNA, or mRNA.

The provided uses and methods may exploit the Gm6A specificity of the provided antibodies. Therefore, when performing a use or method provided herein on one or more samples, there may not be a requirement for the sample to have been subjected to RNA purification step(s) to remove all or substantially all ribosomal RNA (rRNA) from the sample. Alternatively viewed, the method or use may be performed on a sample that may comprise messenger RNA (mRNA) and ribosomal RNA (rRNA). The sample may comprise or consist of total RNA, meaning that it has not been enriched for a particular RNA species and may thus comprise any of the following: mRNA, rRNA, tRNA, circRNA, miRNA, lncRNA, or any other RNA transcript derived from a cell, or an unprocessed primary RNA transcript thereof. Alternatively, the sample may be at least partially depleted of rRNA, for example it may have been depleted of at least 40, 50, 60, 70, 80, 90 or 95% of the rRNA that was originally present. Thus, the RNA content of the sample may include residual rRNA.

Alternatively or in addition, the sample may be enriched for mRNA generally, or be enriched for one or more specific mRNA sequences, such as 1-20, 1-10, 1-5 or 1-2 different specific mRNAs or a single specific mRNA sequence.

Thus, rRNA may represent up to about 30, 20, 10, 9, 8, 7, 6, 5, 4, 3, 2, or 1% of the RNA content of the sample, whilst mRNA represent at least 30, 40, 50 60, 70, 80, 90, 95 or 99% of the RNA content of the sample.

Where a sample is analysed to investigate one or more of its characteristics, the sample may be referred to as a “test sample”. The detection, analysis or quantification of Gm6A may be made relative to a control. A “control” or “control sample” or a “reference sample” is a sample having a known or defined characteristic, that may be used in comparison against a test sample, to allow for characterization of the test sample. Thus, a control will have some characterized property that serves as the basis for comparison with the test sample. For instance, a “control sample” may be substantially free of Gm6A; or a reference sample may be used as a benchmark for Gm6A levels that are indicative of a subject having cancer. The reference sample does not necessarily have to be analyzed in parallel with the test sample, thus in some instances the reference sample may be a numerical value or range or threshold previously determined to characterize a given condition, such as Gm6A that are indicative of cancer in a subject. The term also includes samples used for comparative purposes that are known to be associated with a physiologic state or disease condition, such as a cancer with aberrant expression or activity of a regulator of m6A RNA methylation, but that have unknown levels of Gm6A.

The uses and methods may involve binding a target comprising Gm6A by an antibody or conjugate provided herein. This allows inter alia the purification or detection of a target comprising Gm6A. More particularly, the uses and methods may involve detecting, analyzing, monitoring and/or quantifying the levels of Gm6A in a sample. This may include detecting, analyzing, monitoring and/or quantifying the total levels of Gm6A present in the sample, or the levels of Gm6A within one or more specific nucleic acid molecules. The uses and methods may involve the analysis of 2 or more samples taken (e.g. from a subject or group of subjects) at different time points to monitor temporal changes in Gm6A levels. Alternatively or in addition, 2 or more different samples, e.g. taken from different subjects or different groups of subjects, such as test subjects and healthy controls, may be used to analyse differences of Gm6A levels.

The methods and uses may typically comprise the steps of contacting a subject or a test sample with an antibody or a conjugate provided herein, under conditions effective to allow the formation of complexes between the Gm6A (or Gm6A-containing target) and the antibody or conjugate; and detecting or measuring the presence, amount and/or location of antibody-antigen complex in the subject or test sample.

The provided methods and uses may take the form of an immunofluorescence assay; ELISA; or immunoprecipitation and sequencing, such as Methylated RNA immunoprecipitation sequencing (MeRIP-seq) of m6A individual-nucleotide-resolution cross-linking and immunoprecipitation (miCLIP).

Also provided are corresponding kits comprising the provided antibodies, conjugates, nucleic acids, compositions or the like, e.g. an ELISA kit, an immunofluorescence assay kit, or an immunoprecipitation kit, that may comprise further components required for ELISA, an immunofluorescence assay, or an immunoprecipitation assay.

In some embodiments, the methods and uses further comprise measuring gene expression in the test sample or in a suitable alternative sample, for example a sample derived from the same or a similar source as the test sample. The gene may be directly related to Gm6A or indirectly related to altered Gm6A. For example, gene expression of one or more m6A writers, erasers and/or readers may be measured.

As Gm6A is a dynamic mark in mRNA, without wishing to be bound by theory, the uses and methods provided herein are applicable to monitoring physiological processes or RNA metabolism that directly or indirectly affect Gm6A levels and/or are directly or indirectly impacted by Gm6A levels, particularly aberrant Gm6A levels.

Thus, in further aspects, provided are methods and uses relating to diagnosis, preventing, alleviating or treating a disease, which methods and uses employ the antibodies, conjugates or pharmaceutical compositions provided herein. The discussion herein regarding uses of the provided antibodies and/or conjugates and related methods for binding, detecting, analyzing, monitoring or quantifying Gm6A apply mutatis mutandis to the methods and uses relating to diagnosis, preventing, alleviating or treating a disease.

For example, there is provided an (in vivo, ex vivo or in vitro) method of diagnosing a disease associated with aberrant Gm6A, comprising the steps of contacting a subject or a test sample provided or derived from the subject with an antibody or a conjugate provided herein, under conditions effective to allow the formation of complexes between the Gm6A and the antibody or conjugate; and measuring or detecting the presence, amount and/or location of antibody-antigen complex in the subject or test sample.

Optionally, the method further comprises comparing the presence, amount and/or location of antibody-antigen complex in the subject or test sample to a control. In some embodiments, an increased amount of Gm6A in the test sample is diagnostic for a pre-cancerous or cancerous state, such as leukaemia.

Also provided is a provided antibody, conjugate or pharmaceutical composition, for use in the diagnosis of a disease, disorder and/or abnormality associated with aberrant Gm6A.

Also provided is the use of an antibody provided herein in the manufacture of an imaging or diagnostic agent for the imaging or diagnosis of a disease, disorder and/or abnormality associated with aberrant Gm6A.

The term “treating” refers to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

The skilled person understands that the application of antibodies, conjugates, or pharmaceutical compositions is at an effective amount. The “effective amount” of the pharmaceutical compound which is to be administered to a subject is the dosage which according to sound medical judgement is suitable for treating, preventing or alleviating the disease, disorder or abnormality. The specific dose level and frequency of dosage can depend, e.g., upon a variety of factors including the activity of the specific compound employed, the metabolic stability and length of action of that compound, mode and time of administration. The “effective amount” of the pharmaceutical compound which is to be administered to a subject is the dosage which according to sound medical judgement is suitable for treating, preventing or alleviating the disorder, disease, disorder or abnormality. The specific dose level and frequency of dosage can depend, e.g., upon a variety of factors including the activity of the specific compound employed, the metabolic stability and length of action of that compound, mode and time of administration, the rate of excretion, and drug combination. Patient-specific factors such as the age, body weight, general health, sex, diet, as well as the severity of the particular condition can also influence the amount which is to be administered. Patient-specific factors such as the age, body weight, general health, sex, diet, as well as the severity of the particular condition can also influence the amount which is to be administered.

There is provided a method for preventing, alleviating or treating a disease associated with aberrant Gm6A in a subject, comprising administering to a subject with the disease a therapeutically effective amount of the provided antibody, conjugate, or pharmaceutical composition.

Also provided is a provided antibody, conjugate or pharmaceutical composition, for use in the prevention, alleviation or treatment of a disease, disorder and/or abnormality associated with aberrant Gm6A.

In one aspect, there is provided the use of an antibody provided herein in the manufacture of a medicament for preventing, alleviating or treating a disease, disorder and/or abnormality associated with aberrant Gm6A.

Also provided is a provided antibody or conjugate for use in therapy, imaging or diagnosis, preferably wherein the therapy, imaging or diagnosis is of a disease, disorder and/or abnormality associated with aberrant Gm6A.

The term “disease” used herein, in the context of diagnosis or therapy, may be a disease associated with aberrant Gm6A. The term “aberrant Gm6A” may include (i) increased levels of Gm6A in the total transcriptome; in a fraction thereof; in the transcription products of a specific family or set of genes; or in the transcription products of a specific gene; (ii) decreased levels of Gm6A in the total transcriptome; in a fraction thereof; in the transcription products of a specific family or set of genes; or in the transcription products of a specific gene; (iii) increased levels of Gm6A in the transcription products of a first specific family or set of genes; or in the transcription products of a first specific gene; and decreased levels of Gm6A in the transcription products of a second specific family or set of genes; or in the transcription products of a second specific gene; and/or (iv) an altered pattern of methylation giving rise to the presence of Gm6A in positions that are not methylated in normal controls, and/or giving rise to the absence of Gm6A in positions that are methylated in normal controls (without necessarily altering the levels of Gm6A in that molecule and/or in the transcriptome).

“aberrant Gm6A” may be caused by aberrant expression and/or activity of one or more agents selected from (a) an m6A writer; (b) an m6A eraser; and/or (c) an m6A reader. Examples of m6A writers, m6A erasers, and m6A readers are provided elsewhere herein and are well known in the literature.

Thus, a disease associated with aberrant Gm6A may be a disease associated with, or mediated by, an m6A writer, an m6A eraser, and/or m6A reader, such as aberrant expression and/or activity by an m6A writer, an m6A eraser and/or an m6A reader. The disease associated with aberrant Gm6A may, for example, be a disease associated with or mediated by aberrant expression and/or activity of METTL3.

The disease may, for example, be a cancer, psychiatric disorder, metabolic syndrome or cardiovascular disease, preferably cancer, wherein the cancer is preferably selected from a haematopoietic and a non-haematopoietic cancer.

### I. Numbered Clauses

The invention will now be described by way of numbered clauses.
1. An antibody that is selective for Guanine-N6-methyladenine (Gm6A) over Adenine-N6-methyladenine (A-m6A).
2. The antibody according to clause 1, wherein the antibody is further selective for Gm6A over Cytosine-N6-methyladenine (Cm6A).
3. The antibody according to clause 1 or 2, wherein the antibody is further selective for Gm6A over N6-methyladenine (m6A), N6 N6 dimethyladenine (m6, 6A), N1-methylguanine (m1G), and/or N2,N2-dimethylguanine (m22G).
4. The antibody according to any preceding clause, wherein the antibody is further selective for Gm6A over N1-methyladenine (m1A).
5. The antibody according to any preceding clause, wherein the m6A is in the form of N6-methyladenosine, preferably wherein the Gm6A is in the form of Guanosine-N6-methyladenosine.
6. The antibody according to any preceding clause, wherein the Gm6A is in the form of guanosine linked to N6-methyladenosine via a phosphodiester bond between the ribose moieties and is preferably part of an RNA molecule.
7. The antibody according to any one of clauses 1-4, wherein the m6A is in the form of N6-methyldeoxyadenosine, preferably wherein the Gm6A is in the form of deoxyguanosine-N6-methyldeoxyadenosine.
8. The antibody according to clause 7, where in the Gm6A is in the form of deoxyguanosine linked to N6-methyldeoxyadenosine via a phosphodiester bond between the deoxyribose moieties and is preferably part of a DNA molecule.
9. The antibody according to any preceding clause, wherein the antibody binds Gm6A, wherein the binding is preferably specific.
10. The antibody according to any preceding clause, wherein the antibody comprises:
   (i) a Heavy Chain Variable Region comprising a variable heavy (VH) CDR1 that has the amino acid sequence of SEQ ID NO: 1 or a sequence substantially homologous thereto;
   (ii) a Heavy Chain Variable Region comprising a VH CDR2 that has the amino acid sequence of SEQ ID NO: 2 or a sequence substantially homologous thereto;
   (iii) a Heavy Chain Variable Region comprising a VH CDR3 that has the amino acid sequence of SEQ ID NO: 3 or a sequence substantially homologous thereto;
   (iv) a Light Chain Variable Region comprising a variable light (VL) CDR1 that has the amino acid sequence of SEQ ID NO: 4 or a sequence substantially homologous thereto;
   (v) a Light Chain Variable Region comprising a VL CDR2 that has the amino acid sequence of SEQ ID NO: 5 or a sequence substantially homologous thereto; and/or
   (vi) a Light Chain Variable Region comprising a VL CDR3 that has the amino acid sequence of SEQ ID NO: 6 or a sequence substantially homologous thereto.
11. The antibody according to any preceding clause, wherein the antibody comprises a Heavy Chain Variable Region comprising:
   (i) a variable heavy (VH) CDR1 that has the amino acid sequence of SEQ ID NO: 1 or a sequence substantially homologous thereto;
   (ii) a VH CDR2 that has the amino acid sequence of SEQ ID NO: 2 or a sequence substantially homologous thereto; and
   (iii) a VH CDR3 that has the amino acid sequence of SEQ ID NO: 3 or a sequence substantially homologous thereto.
12. The antibody according to any preceding clause, wherein the antibody comprises a Light Chain Variable Region comprising:
   (iv) a variable light (VL) CDR1 that has the amino acid sequence of SEQ ID NO: 4 or a sequence substantially homologous thereto;
   (v) a VL CDR2 that has the amino acid sequence of SEQ ID NO: 5 or a sequence substantially homologous thereto; and
   (vi) a VL CDR3 that has the amino acid sequence of SEQ ID NO: 6 or a sequence substantially homologous thereto.
13. The antibody according to any preceding clause, wherein the antibody comprises:
   a. a Heavy Chain Variable Region comprising:
   (i) a variable heavy (VH) CDR1 that has the amino acid sequence of SEQ ID NO: 1 or a sequence substantially homologous thereto;
   (ii) a VH CDR2 that has the amino acid sequence of SEQ ID NO: 2 or a sequence substantially homologous thereto;
   (iii) a VH CDR3 that has the amino acid sequence of SEQ ID NO: 3 or a sequence substantially homologous thereto; and
   b. a Light Chain Variable Region comprising:
   (iv) a variable light (VL) CDR1 that has the amino acid sequence of SEQ ID NO: 4 or a sequence substantially homologous thereto;
   (v) a VL CDR2 that has the amino acid sequence of SEQ ID NO: 5 or a sequence substantially homologous thereto;
   (vi) a VL CDR3 that has the amino acid sequence of SEQ ID NO: 6 or a sequence substantially homologous thereto.
14. The antibody according to any one of clauses 10-13, wherein the substantially homologous sequence is a sequence containing exactly or no more than 5, 4, 3, 2 or 1 amino acid substitutions of the given CDR sequence.
15. The antibody according to any one of clauses 10-14, wherein the antibody comprises exactly or no more than 10, 9, 8, 7, 6, 5, 4, 3, 2 or 1 amino acid substitutions in total across all 6 CDRs compared to the recited CDR sequences.
16. The antibody according to any one of clauses 14-15, wherein each substitution, if present, is a conservative amino acid substitution.
17. The antibody according to any preceding clause, wherein the antibody comprises a Heavy Chain Variable Region comprising:
   (i) a VH CDR1 that has the amino acid sequence of SEQ ID NO: 1;
   (ii) a VH CDR2 that has the amino acid sequence of SEQ ID NO: 2; and
   (iii) a VH CDR3 that has the amino acid sequence of SEQ ID NO: 3.
18. The antibody according to any preceding clause wherein the antibody comprises a Light Chain Variable Region comprising:
   (iv) a VL CDR1 that has the amino acid sequence of SEQ ID NO: 4;
   (v) a VL CDR2 that has the amino acid sequence of SEQ ID NO: 5; and
   (vi) a VL CDR3 that has the amino acid sequence of SEQ ID NO: 6.
19. The antibody according to any preceding clause, wherein the antibody comprises:
   a. a Heavy Chain Variable Region comprising:
   (i) a variable heavy (VH) CDR1 that has the amino acid sequence of SEQ ID NO: 1;
   (ii) a VH CDR2 that has the amino acid sequence of SEQ ID NO: 2;
   (iii) a VH CDR3 that has the amino acid sequence of SEQ ID NO: 3; and
   b. a Light Chain Variable Region comprising:
   (iv) a variable light (VL) CDR1 that has the amino acid sequence of SEQ ID NO: 4;
   (v) a VL CDR2 that has the amino acid sequence of SEQ ID NO: 5; and
   (vi) a VL CDR3 that has the amino acid sequence of SEQ ID NO: 6.
20. The antibody according to any preceding clause, which comprises a Heavy Chain Variable Region comprising the sequence of SEQ ID NO: 7 or a Heavy Chain Variable Region having at least 70%, 75%, 80%, 85%, 90% 91%, 92%, 93%, 94%, 95%, 96%, 97%, 98% or 99% identity thereto.
21. The antibody according to any preceding clause, which comprises a Light Chain Variable Region comprising the sequence of SEQ ID NO: 8 or a Light Chain Variable Region having at least 70%, 75%, 80%, 85%, 90% 91%, 92%, 93%, 94%, 95%, 96%, 97%, 98% or 99% identity thereto.
22. The antibody according to any preceding clause, particularly clause 13, which comprises:
   (i) a Heavy Chain Variable Region comprising the sequence of SEQ ID NO: 7 or a Heavy Chain Variable Region having at least 70%, 75%, 80%, 85%, 90% 91%, 92%, 93%, 94%, 95%, 96%, 97%, 98% or 99% identity thereto; and
   (ii) a Light Chain Variable Region comprising the sequence of SEQ ID NO: 8 or a Light Chain Variable Region having at least 70%, 75%, 80%, 85%, 90% 91%, 92%, 93%, 94%, 95%, 96%, 97%, 98% or 99% identity thereto.
23. The antibody according to any preceding clause, which comprises a Heavy Chain Variable Region comprising the sequence of SEQ ID NO: 7 and a Light Chain Variable Region comprising the sequence of SEQ ID NO: 8.
24. The antibody according to any one of clauses 1-9, which comprises:
   a. one, two or all three of the variable heavy (VH) CDR1, VH CDR2 and VH CDR3 of the amino acid sequence of SEQ ID NO: 7; and
   b. one, two or all three of the variable light (VL) CDR1, VL CDR2 and VL CDR3 of the amino acid sequence of SEQ ID NO: 8,
   wherein the CDR sequences are determined in accordance with the Kabat method.
25. The antibody according to clause 24, which comprises:
   a. the VH CDR1, VH CDR2 and VH CDR3 of the amino acid sequence of SEQ ID NO: 7; and
   b. the VL CDR1, VL CDR2 and VL CDR3 of the amino acid sequence of SEQ ID NO: 8,
   wherein the CDR sequences are determined in accordance with the Kabat method.
26. The antibody according to any preceding clause, that binds an epitope comprising or consisting of Gm6A, preferably consisting of Gm6A.
27. The antibody according to any preceding clause, wherein the Gm6A is within an RNA molecule or a DNA molecule, preferably an RNA molecule.
28. The antibody according to any preceding clause, wherein the antibody
   (a) binds Gm6A;
   (b) has a Heavy Chain Variable Region comprising:
      a variable heavy (VH) CDR1 that has the amino acid sequence of SEQ ID NO: 1;
      a VH CDR2 that has the amino acid sequence of SEQ ID NO: 2; and
      a VH CDR3 that has the amino acid sequence of SEQ ID NO: 3; and
   (c) has a Light Chain Variable Region comprising:
      a variable light (VL) CDR1 that has the amino acid sequence of SEQ ID NO: 4;
      a VL CDR2 that has the amino acid sequence of SEQ ID NO: 5; and
      a VL CDR3 that has the amino acid sequence of SEQ ID NO: 6.
29. The antibody according to clause 28, wherein the Gm6A is
   (i) in the form of guanosine linked to N6-methyladenosine via a phosphodiester bond between the ribose moieties and is preferably part of an RNA molecule; or
   (ii) in the form of deoxyguanosine linked to N6-methyldeoxyadenosine via a phosphodiester bond between the deoxyribose moieties and is preferably part of a DNA molecule.
30. The antibody according to any preceding clause, wherein the antibody is selective for GGm6AC over Am6AC.
31. The antibody according to any preceding clause, that is selective for Gm6A within messenger RNA (mRNA) over Am6A within ribosomal RNA (rRNA).
32. The antibody according to clause 31, wherein the rRNA is 28S and/or 18S rRNA, preferably 28S and 18S rRNA.
33. The antibody according to clause 31 or 32, wherein the RNA molecule is single stranded (ssRNA).
34. The antibody according to any of clauses 31 to 33, wherein the RNA molecule is messenger RNA (mRNA).
35. The antibody according to any of clauses 31 to 34, wherein the RNA molecule is prokaryotic or eukaryotic.
36. The antibody according to clause 35, wherein the RNA molecule is obtained from human or mouse.
37. An antibody that is selective for messenger RNA (mRNA) comprising m6A over ribosomal RNA (rRNA) comprising m6A, wherein the antibody is preferably according to any preceding clause.
38. The antibody according to any preceding clause, wherein selectivity is determined by one or more of:
(i) dot blot;
(ii) homogeneous time-resolved fluorescence (HTRF); and/or
(iii) immunocytochemistry (ICC).
39. An antibody that specifically binds Gm6A, wherein the antibody preferably has the 6 CDR sequences of SEQ ID NOs 1-6.
40. An antibody according to clause 1, wherein the antibody can compete for binding to Gm6A with the antibody of any one of clauses 2 to 38, preferably clause 19, 23, 28 or 29, for example wherein antibody can compete for binding to Gm6A with the specific Gm6A-selective antibody exemplified herein, 21H1L4.
41. The antibody according to clause 40, wherein the antibody binds to substantially the same epitope, or to the same epitope, as the antibody defined in clause 19, 23, 28 or 29, for example wherein the antibody can compete for binding to Gm6A with the specific Gm6A-selective antibody exemplified herein, 21H1L4.
42. The antibody according to clause 40 or 41, wherein the antibody is functionally equivalent to the antibody of any one of clauses 2 to 38, preferably to the antibody of clause 19, 23, 28 or 29, for example wherein the antibody is functionally equivalent to the specific Gm6A-selective antibody exemplified herein.
43. The antibody according any one of clauses 40-42, wherein the antibody comprises:
   (i) a Heavy Chain Variable Region comprising a variable heavy (VH) CDR1 that has the amino acid sequence of SEQ ID NO: 1 or a sequence substantially homologous thereto; (ii) a Heavy Chain Variable Region comprising a VH CDR2 that has the amino acid sequence of SEQ ID NO: 2 or a sequence substantially homologous thereto;
   (iii) a Heavy Chain Variable Region comprising a VH CDR3 that has the amino acid sequence of SEQ ID NO: 3 or a sequence substantially homologous thereto;
   (iv) a Light Chain Variable Region comprising a variable light (VL) CDR1 that has the amino acid sequence of SEQ ID NO: 4 or a sequence substantially homologous thereto;
   (v) a Light Chain Variable Region comprising a VL CDR2 that has the amino acid sequence of SEQ ID NO: 5 or a sequence substantially homologous thereto; and/or
   (vi) a Light Chain Variable Region comprising a VL CDR3 that has the amino acid sequence of SEQ ID NO: 6 or a sequence substantially homologous thereto.
44. The antibody according to any one of clauses 40 to 43, wherein the antibody comprises:
   a. a Heavy Chain Variable Region comprising:
   (i) a variable heavy (VH) CDR1 that has the amino acid sequence of SEQ ID NO: 1 or a sequence substantially homologous thereto;
   (ii) a VH CDR2 that has the amino acid sequence of SEQ ID NO: 2 or a sequence substantially homologous thereto;
   (iii) a VH CDR3 that has the amino acid sequence of SEQ ID NO: 3 or a sequence substantially homologous thereto; and/or
   b. a Light Chain Variable Region comprising:
   (iv) a variable light (VL) CDR1 that has the amino acid sequence of SEQ ID NO: 4 or a sequence substantially homologous thereto;
   (v) a VL CDR2 that has the amino acid sequence of SEQ ID NO: 5 or a sequence substantially homologous thereto;
   (vi) a VL CDR3 that has the amino acid sequence of SEQ ID NO: 6 or a sequence substantially homologous thereto.
45. The antibody according to clauses any one of clauses 40 to 43, wherein the antibody comprises a Heavy Chain Variable Region comprising:
   (i) a variable heavy (VH) CDR1 that has the amino acid sequence of SEQ ID NO: 1 or a sequence substantially homologous thereto;
   (ii) a VH CDR2 that has the amino acid sequence of SEQ ID NO: 2 or a sequence substantially homologous thereto; and
   (iii) a VH CDR3 that has the amino acid sequence of SEQ ID NO: 3 or a sequence substantially homologous thereto.
46. The antibody according to clauses any one of clauses 40 to 43, wherein the antibody comprises a Light Chain Variable Region comprising:
   (iv) a variable light (VL) CDR1 that has the amino acid sequence of SEQ ID NO: 4 or a sequence substantially homologous thereto;
   (v) a VL CDR2 that has the amino acid sequence of SEQ ID NO: 5 or a sequence substantially homologous thereto; and
   (vi) a VL CDR3 that has the amino acid sequence of SEQ ID NO: 6 or a sequence substantially homologous thereto.
47. The antibody according to clauses any one of clauses 40 to 43, wherein the antibody comprises:
   a. a Heavy Chain Variable Region comprising:
   (i) a variable heavy (VH) CDR1 that has the amino acid sequence of SEQ ID NO: 1 or a sequence substantially homologous thereto;
   (ii) a VH CDR2 that has the amino acid sequence of SEQ ID NO: 2 or a sequence substantially homologous thereto;
   (iii) a VH CDR3 that has the amino acid sequence of SEQ ID NO: 3 or a sequence substantially homologous thereto; and
   b. a Light Chain Variable Region comprising:
   (iv) comprising a variable light (VL) CDR1 that has the amino acid sequence of SEQ ID NO: 4 or a sequence substantially homologous thereto;
   (v) a VL CDR2 that has the amino acid sequence of SEQ ID NO: 5 or a sequence substantially homologous thereto;
   (vi) a VL CDR3 that has the amino acid sequence of SEQ ID NO: 6 or a sequence substantially homologous thereto.
48. The antibody according to clause 1, wherein the antibody
   (a) Binds Gm6A, preferably an epitope comprising or consisting of Gm6A;
   (b) can compete for binding to Gm6A with the antibody of any one of clauses 2 to 38, preferably clause 19, 23, 28 or 29, for example wherein antibody can compete for binding to Gm6A with the specific Gm6A-selective antibody exemplified herein;
   (c) comprises a Heavy Chain Variable Region comprising:
   (i) a variable heavy (VH) CDR1 that has the amino acid sequence of SEQ ID NO: 1 or a sequence substantially homologous thereto;
   (ii) a VH CDR2 that has the amino acid sequence of SEQ ID NO: 2 or a sequence substantially homologous thereto;
   (iii) a VH CDR3 that has the amino acid sequence of SEQ ID NO: 3 or a sequence substantially homologous thereto; and
   (d) comprises a Light Chain Variable Region comprising:
   (iv) comprising a variable light (VL) CDR1 that has the amino acid sequence of SEQ ID NO: 4 or a sequence substantially homologous thereto;
   (v) a VL CDR2 that has the amino acid sequence of SEQ ID NO: 5 or a sequence substantially homologous thereto;
   (vi) a VL CDR3 that has the amino acid sequence of SEQ ID NO: 6 or a sequence substantially homologous thereto.
49. The antibody according to clause 48, wherein the Gm6A is
   (i) in the form of guanosine linked to N6-methyladenosine via a phosphodiester bond between the ribose moieties and is preferably part of an RNA molecule; or
   (ii) in the form of deoxyguanosine linked to N6-methyldeoxyadenosine via a phosphodiester bond between the deoxyribose moieties and is preferably part of a DNA molecule.
50. The antibody according to any one of clauses 43-49, wherein each substantially homologous sequence is a sequence containing exactly or no more than 5, 4, 3, 2 or 1 amino acid changes of the given CDR sequence, wherein the amino acid changes are selected from substitutions, additions and/or deletions.
51. The antibody according to any one of clauses 43-50, wherein said antibody comprises exactly or no more than 30, 25, 20, 19, 18, 17, 16, 15, 14, 13, 12, 11, 10, 9, 8, 7, 6, 5, 4, 3, 2 or 1 amino acid changes in total across all 6 CDRs compared to the recited CDR sequences, wherein the amino acid changes are selected from substitutions, additions and/or deletions.
52. The antibody according to any one of clauses 50-51, wherein the amino acid changes are substitutions.
53. The antibody according clause 21, wherein each substitution, if present, is a conservative amino acid substitution.
54. The antibody according to any one of clauses 40 to 53, which comprises a Heavy Chain Variable Region comprising the sequence of SEQ ID NO: 7 or a Heavy Chain Variable Region having at least 70%, 75%, 80%, 85%, 90% 91%, 92%, 93%, 94%, 95%, 96%, 97%, 98% or 99% identity thereto.
55. The antibody according to any one of clauses 40 to 53, which comprises a Light Chain Variable Region comprising the sequence of SEQ ID NO: 8 or a Light Chain Variable Region having at least 70%, 75%, 80%, 85%, 90% 91%, 92%, 93%, 94%, 95%, 96%, 97%, 98% or 99% identity thereto.
56. The antibody according to any one of clauses 40 to 55, which comprises:
   (i) a Heavy Chain Variable Region comprising the sequence of SEQ ID NO: 7 or a Heavy Chain Variable Region having at least 70%, 75%, 80%, 85%, 90% 91%, 92%, 93%, 94%, 95%, 96%, 97%, 98% or 99% identity thereto; and
   (ii) a Light Chain Variable Region comprising the sequence of SEQ ID NO: 8 or a Light Chain Variable Region having at least 70%, 75%, 80%, 85%, 90% 91%, 92%, 93%, 94%, 95%, 96%, 97%, 98% or 99% identity thereto.
57. The antibody according to any preceding clause, wherein the antibody is a monoclonal antibody.
58. The antibody according to any preceding clause, wherein the antibody is isolated.
59. The antibody according to any preceding clause, wherein the antibody is recombinant.
60. The antibody according to any preceding clause, wherein the antibody is chimeric.
61. The antibody according to any preceding clause, wherein the antibody is humanized or fully human.
62. The antibody according to any preceding clause, wherein the antibody is an IgA, IgD, IgE, IgM, or IgG antibody.
63. The antibody according to clause 62, wherein the antibody is an IgG antibody.
64. The antibody according to any preceding clause, wherein the antibody is glycosylated.
65. The antibody according to any preceding clause, wherein the antibody is a bivalent or polyvalent antibody comprising at least two antigen binding fragments of the antibody.
66. The antibody according to any preceding clause, wherein the antibody is an antigen-binding fragment of an antibody.
67. The antibody of clause 64, wherein the antigen binding fragment of the antibody is a Fab, F(ab′)₂, Fv, scFv, diabody, monovalent IgG, scFv-Fc, minibody, or triabody.
68. The antibody according to any preceding clause further comprising a detectable label.
69. A conjugate comprising the antibody of any of the preceding clauses, preferably clause 19, 23, 28, 29 or 40.
70. The conjugate of clause 69, wherein the antibody is conjugated to an agent, wherein the agent is preferably a selectable tag, a detectable label, a toxin, or a therapeutic agent.
71. The conjugate of clause 70, wherein the agent comprises or consists of an enzyme, avidin, streptavidin, biotin, Protein A, Protein G, magnetic beads, fluorophores, metal, radioactive isotopes nucleic acid molecules, or a toxin.
72. The conjugate of clause 70, wherein the agent is a therapeutic agent that comprises or consists of a chemotherapeutic agent, drug, growth inhibitory agent, toxin, radiotherapeutic agent, anti-angiogenic agent, apoptosis-inducing agent, anti-tubulin drug, cytotoxic agent, steroid, cytokine antagonist, cytokine expression inhibitor, targeted protein, RNA degradation agent or chemokine antagonist.
73. A composition comprising an antibody according to any of clauses 1 to 68 or a conjugate according to any of clauses 69 to 72.
74. A pharmaceutical composition comprising the antibody of any of clauses 1 to 68 or a conjugate according to any of clauses 69 to 72, and a pharmaceutically acceptable carrier and/or excipient.
75. A nucleic acid molecule comprising a nucleotide sequence region, wherein the nucleotide sequence region
   (a) encodes the antibody of any one of clauses 1 to 68, particularly any one of clauses 10-25 or 43-48; and/or
   (b) has the nucleotide sequence of SEQ ID NO: 9 and/or SEQ ID NO: 10, or a sequence having at least 70%, 75%, 80%, 85%, 90%, 95%, 96%, 97%, 98% or at least 99% identity to SEQ ID NO: 9 and/or SEQ ID NO: 10, for example comprises SEQ ID NO: 9 and/or SEQ ID NO: 10.
76. A nucleic acid molecule comprising a nucleotide sequence region (a) encoding a variable heavy chain region and/or a variable light chain region of the antibody of any of clauses 1 to 68, particularly any one of clauses 10-25 or 43-48; and/or (b) having the nucleotide sequence of SEQ ID NO: 9 and/or SEQ ID NO: 10, or a sequence having at least 70%, 75%, 80%, 85%, 90%, 95%, 96%, 97%, 98% or at least 99% identity to SEQ ID NO: 9 and/or SEQ ID NO: 10, for example comprising SEQ ID NO: 9 and/or SEQ ID NO: 10.
77. A set of nucleic acid molecules, comprising:
   (i) a nucleic acid molecule comprising or consisting of a nucleotide sequence region encoding a variable heavy chain region, of the antibody of any of clauses 1 to 68, particularly any one of clauses 10-25 or 43-48, for example comprising or consisting of SEQ ID NO: 9; and
   (ii) a nucleic acid molecule comprising or consisting of a nucleotide sequence region encoding a variable light chain region, of the antibody of any of clauses 1 to 68, particularly any one of clauses 10-25 or 43-48, for example comprising or consisting of SEQ ID NO: 10.
78. An expression vector comprising the nucleic acid molecule according to any of clauses 75 to 76, or the set of nucleic acid molecules according to clause 77.
79. An expression vector, or a set of expression vectors, comprising one or more nucleic acid molecules of clause 77, wherein the expression vector, or set of expression vectors, encodes and is suitable for expression of the antibody of any of clauses 1 to 68, particularly any one of clauses 10-25 or 43-48 or a Heavy Chain Variable Region and/or a Light Chain Variable Region thereof.
80. A set of expression vectors, comprising
   (a) a first expression vector comprising the nucleic acid molecule (i) of clause 77(i); and
   (b) a second expression vector comprising the nucleic acid molecule (ii) of clause 77(ii).
81. A cell-free expression system containing the expression vector according to clause 78 or 79 or set of expression vectors according to clause 79 or 80.
82. A host cell comprising the nucleic acid molecule according to any one of clauses 75 to 76, the set of nucleic acid molecules according to clause 77, the expression vector according to clause 78 or 79 and/or the set of expression vectors according to clause 79 or 80.
83. A virus comprising the nucleic acid molecule according to any of clauses 75 to 76, the set of nucleic acid molecules according to clause 77, the expression vector according to clause 78 or 79 and/or the set of expression vectors according to clause 79 or 80.
84. A kit, optionally in one or more containers, comprising the:
   (a) antibody according to any of clauses 1 to 68;
   (b) conjugate according to any of clauses 69 to 72;
   (c) composition according to clause 73;
   (d) pharmaceutical composition according to clause 74;
   (e) nucleic acid molecule according to any of clauses 75 to 76;
   (f) set of nucleic acid molecules according to clause 77;
   (g) expression vector according to clause 78 or 79;
   (h) set of expression vectors according to clause 79 or 80;
   (i) host cell according to clause 82; and/or
   (j) virus according to clause 83;
and further comprising a:
   (k) detectable label; and/or
   (l) therapeutic agent,
   wherein the kit may optionally further be an ELISA kit, an immunofluorescence assay kit, or an immunoprecipitation kit.
85. The kit according to clause 84 for detecting Gm6A, for example for monitoring or diagnosis of a disease, disorder and/or abnormality associated with aberrant Gm6A.
86. A method of producing an antibody, comprising:
   (a) culturing a host cell that comprises the expression vector according to clause 78 or 79, or set of expression vectors according to clause 79 or 80, under conditions effective to express the encoded antibody; and
   (b) obtaining the expressed antibody.
87. The method of clause 86, wherein the antibody is according to any one of clauses 1 to 68.
88. Use of an antibody according to any one of clauses 1 to 68 or a conjugate of any one of clauses 69 to 72 in an in vitro method of detecting, analysing or quantifying Gm6A levels in a sample, for example to assess the regulation of m6A modifications in mRNA.
89. A method of binding Gm6A, preferably Gm6A within an RNA molecule, comprising contacting a sample comprising Gm6A with the antibody according to any one of clauses 1 to 68, or an conjugate according to any of clauses 69 to 72.
90. A method of detecting, analysing and/or quantifying Gm6A, preferably Gm6A within an RNA molecule, comprising:
   (a) contacting a sample with the antibody according to any of clauses 1 to 68, or a conjugate according to any of clauses 69 to 72, under conditions effective to allow the formation of complexes between Gm6A and the antibody; and
   (b) detecting, analysing, and/or quantifying the complexes so formed;
   and optionally comparing the results to a control sample.
91. A method of monitoring the levels of adenine methylation, preferably messenger RNA (mRNA) adenine methylation, preferably adenine methylation in one or more GA (motifs), comprising:
   (a) contacting a sample with the antibody according to any of clauses 1 to 68 or an conjugate according to any of clauses 69 to 72, under conditions effective to allow the formation of complexes between Gm6A and the antibody; and
   (b) detecting the complexes so formed;
   (c) optionally obtaining one or more additional samples and repeating steps (a) and (b).
92. The method according to any of clauses 86-91, further comprising measuring gene expression in the sample or a suitable different sample, for example gene expression of one or more m6A writers, erasers and/or readers may be measured.
93. The method according to any of clauses 86 to 92, wherein the sample is sample provided or derived from a human or animal subject, preferably a human subject.
94. The method according to any of clauses 86 to 93, wherein the sample comprises total RNA.
95. The method according to any of clauses 86 to 94, wherein the method does not require, and preferably does not include, an RNA purification step to remove ribosomal RNA (rRNA) from the sample.
96. The method according to any of clauses 86 to 85, wherein the sample comprises messenger RNA (mRNA) and ribosomal RNA (rRNA).
97. The method according to any of clauses 86 to 85, wherein the sample (i) is at least partially depleted of rRNA; and/or (ii) is enriched for mRNA generally, or enriched for one or more specific mRNA sequences, such as 1-20, 1-10, 1-5 or 1-2 different specific mRNAs or a single specific mRNA sequence.
98. The method according to any of clauses 86 to 97, wherein the detected or monitored RNA is single stranded RNA (ssRNA).
99. The method according to any of clauses 86 to 98, wherein the detected or monitored RNA is messenger RNA (mRNA).
100. The method according to any of clauses 86 to 99, wherein the detected or monitored RNA is prokaryotic or eukaryotic.
101. The method according to any of clauses 86 to 100, which is used to monitor a physiological process, such as hematopoietic development, central nervous system development, or reproductive system development, preferably embryonic development, brain development, spermatogenesis, cell reprogramming and/or T cell homeostasis.
102. The method according to any of clauses 86 to 100, which is used to monitor RNA metabolism, such as RNA splicing, RNA translation, RNA stability, RNA translocation, preferably wherein the RNA is mRNA.
103. A method of diagnosing a disease associated with aberrant Gm6A, comprising the steps of:
   (a) contacting a subject or a (test) sample taken from the subject with an antibody according to any of clauses 1 to 68 or a conjugate according to any of clauses 69 to 72, under conditions effective to allow the formation of complexes between the Gm6A and the antibody or conjugate; and
   (b) measuring or detecting the presence, amount and/or location of antibody-antigen complex in the subject or test sample; and optionally
   (c) comparing the presence, amount and/or location of antibody-antigen complex in the subject or test sample to a control.
104. The method according to clause 103, wherein the nucleic acid content of the (test) sample comprises or consists of:
   a. total RNA;
   b. total mRNA;
   c. RNA that is at least partially depleted of rRNA; and/or is enriched for mRNA;
   d. RNA that is at least partially depleted of rRNA and is enriched for mRNA
   e. RNA enriched for one or more specific mRNAs;
   f 1-20, 1-10, 1-5 or 1-2 different specific mRNAs; or
   g. a specific mRNA.
105. The method according to any of clauses 89 to 104, particularly 103-104, wherein an increased amount of Gm6A in the test sample is diagnostic for tumour cells, such as leukaemia.
106. The method according to any of clauses 89 to 105, wherein the subject is a human subject or the (test) sample is from a human subject.
107. A method for preventing, alleviating or treating a disease associated with aberrant Gm6A in a subject, comprising administering to a subject with the disease a therapeutically effective amount of the antibody according to any of clauses 1 to 68, conjugate according to any of clauses 69 to 72, or pharmaceutical composition according to clause 74.
108. The method according to any of clauses 103 to 107, wherein the disease associated with aberrant Gm6A is a disease associated with or mediated by an m6A writer, m6A eraser and/or m6A reader, preferably aberrant expression and/or activity by an m6A writer, m6A eraser and/or m6A reader.
109. The method according to clause 108, wherein:
   (a) the writer is selected from METTL3, METTL14, METTL16, WTAP, KIAA1429, RBM15 and ZC3H13;
   (b) the eraser is selected from FTO and ALKBH5; and/or
   (c) the reader is selected from YTHDC1, HNRNPA2B1, HNRNPC, YTHDF1, YTHDF2, YTHDF3, YTHDC2 and IGF2BP1/2/3,
110. The method according to any of clauses 103 to 109, wherein the disease associated with aberrant Gm6A is a disease associated with or mediated by aberrant expression and/or activity of METTL3.
111. The method according to any of clauses 103 to 110, wherein the disease is a cancer, psychiatric disorder, metabolic syndrome or cardiovascular disease, preferably cancer, wherein the cancer is preferably selected from a haematopoietic or a non-haematopoietic cancer.
112. The method according to any of clauses 107 to 111, further comprising administering a second therapeutic agent to the subject.
113. The method according to any of clauses 107 to 112, wherein the antibody is a bivalent or polyvalent antibody comprising at least two antigen binding fragments of the antibody.
114. The method according to any of clauses 103 to 113, wherein the subject is a human subject.
115. The antibody according to any of clauses 1 to 68, conjugate according to any of clauses 69 to 72, or pharmaceutical composition according to clause 74, for use in therapy, imaging or diagnosis, preferably wherein the therapy, imaging or diagnosis is of a disease, disorder and/or abnormality associated with aberrant Gm6A.
116. The antibody according to any of clauses 1 to 68, conjugate according to any of clauses 69 to 72, or pharmaceutical composition according to clause 74, for use in the prevention, alleviation or treatment of a disease, disorder and/or abnormality associated with aberrant Gm6A.
117. The antibody according to any of clauses 1 to 68, conjugate according to any of clauses 69 to 72, or pharmaceutical composition according to clause 74, wherein the disease, disorder and/or abnormality with aberrant Gm6A is a disease associated with or mediated by an m6A writer, an m6A eraser and/or an m6A reader, preferably aberrant expression and/or activity by an m6A writer, an m6A eraser and/or an m6A reader.
118. The antibody, conjugate or pharmaceutical composition according to clause 117, wherein:
   (a) the writer is selected from METTL3, METTL14, METTL16, WTAP, KIAA1429, RBM15 and ZC3H13;
   (b) the eraser is selected from FTO and ALKBH5; and/or
   (c) the reader is selected from YTHDC1, HNRNPA2B1, HNRNPC, YTHDF1, YTHDF2, YTHDF3, YTHDC2 and IGF2BP1/2/3,
119. The antibody, conjugate or pharmaceutical composition according to any of clauses 115 to 118, wherein the disease associated with aberrant Gm6A is a disease associated with or mediated by aberrant expression and/or activity of METTL3.
120. The antibody, conjugate or pharmaceutical composition according to any of clauses 115 to 119, wherein the disease is a cancer, psychiatric disorder, metabolic syndrome or cardiovascular disease, preferably cancer, wherein the cancer is preferably selected from a haematopoietic and a non-haematopoietic cancer.
121. Use of an antibody according to any of clauses 1 to 68 or conjugate according to any of clauses 69 to 72 in the manufacture of a medicament for preventing, alleviating or treating a disease, disorder and/or abnormality associated with aberrant Gm6A, optionally wherein the disease is a cancer, psychiatric disorder, metabolic syndrome or cardiovascular disease.
122. Use of an antibody according to any one of clauses 1-68 or a conjugate according to any one of clauses 69-71 in an in vitro method of detecting, analysing or quantifying Gm6A levels in a sample.
123. Use of Gm6A as a biomarker of disease, comprising contacting a sample with an antibody according to any of clauses 1 to 68 or a conjugate according to any of clauses 69-72 and comparing the Gm6A levels in the sample to a control.

### BRIEF DESCRIPTION OF THE FIGURES

FIG. 1. Carboxy C2 dG m6A compound used to raise anti-Gm6A antibodies.

FIG. 2. Dot blot assay showing the 21H1L4 antibody binding to RNA samples and synthetic oligonucleotides. Lane 1: total RNA from HeLa cells at 5 μg, 0.5 μg, 0.05 μg and 0.005 μg (top to bottom). Lane 2: HeLa mRNA at 0.4 μg and 0.04 μg (top and bottom). Lane 3: “Gm6A oligonucleotide”. Lane 4: “Am6A oligonucleotide”. Lane 5: “Cm6A oligonucleotide”. Lane 6: “A oligonucleotide”. Lane 7: “m66A oligonucleotide”. See Example 2 for details of the oligonucleotides.

Lanes 3-7 nucleic acid is at 100 ng (top) and 10 ng (bottom) concentrations. Bottom panels show controls: left-hand side lanes 1 and 2 stained with methylene blue to confirm presence of RNA, right-hand side lanes stained with streptavidin-Alexa 488 (lanes 3-7) to confirm presence of biotinylated oligonucleotides. Data shows strong selectivity of antibody 21H1L4 for Gm6A.

FIG. 3. Gradient HTRF showing 21H1L4 antibody selectivity to G-m6A synthetic oligonucleotide. Oligonucleotides tested: “Gm6A”, “A”, “Am6A”, “Cm6A”, and “m66A”. See Example 2 for details of the oligonucleotides. Y axis: % DeltaF, X axis: 21H1L4 antibody concentration (μg/ml). Data shows strong selectivity of antibody 21H1L4 for Gm6A.

FIG. 4. Dot blot assay of antibody 21H1L4 and comparators. Left side: dot blot results for multiple antibodies. Right side: RNA control staining (top is methylene blue, bottom is Cyanine 3). Left panel columns: 1-7 exposed for 5 seconds, then lane 3 repeated—exposed for 64 seconds. Antibodies per (lane):
(1) Ab190886 (comparator recombinantly produced rabbit monoclonal antibody to N6-methyladenosine, available from Abcam Plc catalogue number 190886) used at 1 μg/ml,
(2) Ab151230 (comparator rabbit polyclonal to N6-methyladenosine, available from Abcam Plc catalogue number 151230) used at 1 μg/ml
(3) Ab208577 (comparator mouse monoclonal [17-3-4-1] to N6-methyladenosine, available from Abcam Plc catalogue number 208577) used at 1 μg/ml
(4) Antibody 21H1L4 used at 1 μg/ml
(5) Antibody H3L3 (comparator rabbit monoclonal to N6-Methyladenosine) used at 0.58 μg/ml
(6) Antibody XVJ-1_30-1A (comparator rabbit monoclonal to N6-Methyladenosine) used at 0.5 μg/ml,
(7) CST56593S (comparator rabbit monoclonal to N6-Methyladenosine (D9D9W), available from Cell Signaling Technology (CST) catalogue number #56593) used at 1 μg/ml.

RNA samples—rows 1-11, 1—HeLa total RNA, 2—HeLa mRNA, 3—“A oligonucleotide”, 4—“m66A oligonucleotide”, 5—“Gm6A oligonucleotide”, 6—“Am6A oligonucleotide”, 7—“Cm6A oligonucleotide”, 8—“m1A oligonucleotide”, 9—“m1G oligonucleotide”, 10—“m22G oligonucleotide”, 11—“m6A oligonucleotide”. See Example 2 for details of the oligonucleotides.

FIG. 5. Gradient HTRF showing 21H1L4 antibody selectivity to Gm6A synthetic oligonucleotide. Oligonucleotides tested: “A”, “m66A”, “Gm6A”, “Am6A”, “Cm6A”, “m1A”, “m1G” and “m22G”. See Example 2 for details of the oligonucleotides.

Y axis: % DeltaF, X axis: 21H1L4 antibody concentration (μg/ml). Data shows strong selectivity for Gm6A (top line).

FIG. 6. Dot blots using total RNA from wild type (WT) or Mettl3 knockout mouse embryonic fibroblasts (MEFs) with pan-m6A and 21H1L4 antibodies. 2 μg total RNA from WT and Mettl3 KO MEFs tested to confirm presence of RNA in sample. Left side columns: (1) WT, (2) Mettl3 KO. Right side is control staining with methylene blue. Rows: 1—pan-m6A antibody, 2—Antibody 21H1L4.

FIG. 7. Mouse embryonic fibroblast immunocytochemistry from wild type and Mettl3 knockout cells stained with antibody 21H1L4 and rRNA marker. Antibody 21H1L4 staining was only observed for the wild-type, not for the Mettl3 knockout.

FIG. 8. Chemical formula of m6A.

### EXAMPLES

### Example 1: Antibodies

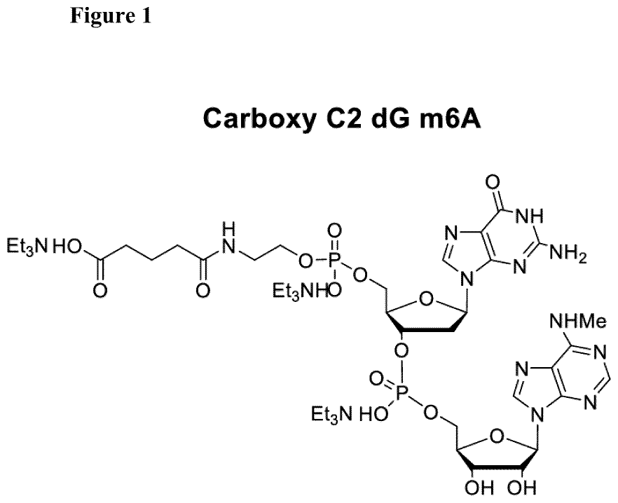

Antibodies were raised against Carboxy C2 dG m6A (FIG. 1). Several antibodies were generated and some of these were able to bind oligonucleotides comprising m6A, but only one of these antibodies, denoted herein 21H1L4, was selective for Gm6A over other methylated nucleotides such as Adenine-N6-methyladenine (Am6A).

Nucleotide sequences of the heavy chain and light chain variable domains (VH and VL) are shown in Table 2. Protein sequences for VH and VL chain variable domains are shown in Table 3 and their complementarity-determining regions (CDRs) are shown in Table 4.

Nucleotide sequence of the heavy chain is shown in Table 5. Nucleotide sequence of the light chain is shown in Table 5. Protein sequence for the heavy chain is shown in Table 6. Protein sequence for the light chain is shown in Table 6.

TABLE 2
Nucleotide sequences of the Heavy Chain and Light Chain
Variable Domains (VH and VL)

| Antibody | | |
|---|---|---|
| Name | Domain | Nucleotide sequence |
| 21H1L4 | VH | ATGGAGACTGGGCTGCGCTGGCTTCTCCTGGTCGCTG |
| | | TGCTCAAAGGTGTCCAGTGTCAGTCGTTGGAGGAGT |
| | | CCGGGGGAGACCTGGTCAAGCCTGGGGCATCCCTGA |
| | | CACTCACCTGCAAAGCCTCTGGATTCTCCTTCGGTAG |
| | | TAGTGCTTGGATATGGTGGGTCCGCCAGGCTCCAGG |
| | | GAAGGGGCTGGAGGGGGTCGCATGGATTTATACTGG |
| | | TGGTGCTGGTAGACTTTACTACGCGAGCTGGGCGAA |
| | | AGGCCGATTCACCATCTCCAAAACCTCGTCGACCAC |
| | | GGTGACTCTGCAAATGACCAGTCTGACAGCCGCGGA |
| | | CACGGCCACCTATTTCTGTGCGAGAAATTCTGCATTG |
| | | TGGGGCCCAGGCACCCTGGTCACCGTCTCCTCA |
| | | (SEQ ID NO: 9) |
| | VL | ATGGACACGAGGGCACCCGCTCAGCTGCTGGGGCTC |
| | | CTGCTGCTCTGGCTCCCAGGTGCCACATTTGCCCAAG |
| | | TGCTGACCCAGACTCCATCCTCCGTGTCTGCAGCTGT |
| | | GGGAGGCACAGTCACCATCAATTGCCAGTCCGGTCA |
| | | GAGTGTTTATAATACCAACCAATTCTCCTGGATTCAG |
| | | CAGAAAGCAGGGCAGCCTCCCAAGTTCCTGATCGCC |
| | | GATGCATCCAAACTGGCATCTGGGGTCCCATCGCGG |
| | | TTCAGCGGCAGTGGATCTGGGACACACTTCACTCTC |
| | | ACCATCAGCGACGTCCAGTGTGACGATGCTGCCACT |
| | | TACTACTGTCTAGGCGGTTATGATTGTAGTAGTGCTG |
| | | ATTGCTCTGCTTTCGGCGGAGGGACCGAGGTGGTGG |
| | | TCGTT |
| | | (SEQ ID NO: 10) |

TABLE 3
Amino acid sequences of the Heavy Chain and Light Chain
Variable Domains (VH and VL)

| Antibody | | |
|---|---|---|
| Name | Domain | Amino Acid sequence |
| 21H1L4 | VH | METGLRWLLLVAVLKGVQCQSLEESGGDLVKPGASLT |
| | | LTCKASGFSFGSSAWIWWVRQAPGKGLEGVAWIYTGG |
| | | AGRLYYASWAKGRFTISKTSSTTVTLQMTSLTAADTAT |
| | | YFCARNSAL WGPGTLVTVSS |
| | | (SEQ ID NO: 7) |
| | VL | MDTRAPAQLLGLLLLWLPGATFAQVLTQTPSSVSAAV |
| | | GGTVTINCQSGQSVYNTNQFSWIQQKAGQPPKFLIADA |
| | | SKLASGVPSRFSGSGSGTHFTLTISDVQCDDAATYYCL |
| | | GGYDCSSADCSAFGGGTEVVVV |
| | | (SEQ ID NO: 8) |

TABLE 4
Amino acid sequences of the Heavy Chain and Light Chain
Variable Domains (VH and VL) and their CDRs

| Antibody | | |
|---|---|---|
| name | Domain | Sequence |
| 21H1L4 | VH CDR 1 | SSAWIW (SEQ ID NO: 1) |
| | VH CDR 2 | WIYTGGAGRLYYASWAKG (SEQ ID NO: 2) |
| | VH CDR3 | NSAL (SEQ ID NO: 3) |
| | VL CDR 1 | QSGQSVYNTNQFS (SEQ ID NO: 4) |
| | VL CDR 2 | DASKLAS (SEQ ID NO: 5) |
| | VL CDR 3 | LGGYDCSSADCSA (SEQ ID NO: 6) |

TABLE 5
Nucleotide sequence of the Heavy Chain (HC) and Light Chain (LC)

| Antibody | | |
|---|---|---|
| Name | Chain | Nucleotide sequence |
| 21H1L4 | HC | ATGGAGACTGGGCTGCGCTGGCTTCTCCTGGTCGCTG |
| | | TGCTCAAAGGTGTCCAGTGTCAGTCGTTGGAGGAGT |
| | | CCGGGGGAGACCTGGTCAAGCCTGGGGCATCCCTGA |
| | | CACTCACCTGCAAAGCCTCTGGATTCTCCTTCGGTAG |
| | | TAGTGCTTGGATATGGTGGGTCCGCCAGGCTCCAGG |
| | | GAAGGGGCTGGAGGGGGTCGCATGGATTTATACTGG |
| | | TGGTGCTGGTAGACTTTACTACGCGAGCTGGGCGAA |
| | | AGGCCGATTCACCATCTCCAAAACCTCGTCGACCAC |
| | | GGTGACTCTGCAAATGACCAGTCTGACAGCCGCGGA |
| | | CACGGCCACCTATTTCTGTGCGAGAAATTCTGCATTG |
| | | TGGGGCCCAGGCACCCTGGTCACCGTCTCCTCAGGG |
| | | CAACCTAAGGCTCCATCAGTCTTCCCACTGGCCCCCT |
| | | GCTGCGGGGACACACCCAGCTCCACGGTGACCCTGG |
| | | GCTGCCTGGTCAAAGGGTACCTCCCGGAGCCAGTGA |
| | | CCGTGACCTGGAACTCGGGCACCCTCACCAATGGGG |
| | | TACGCACCTTCCCGTCCGTCCGGCAGTCCTCAGGCCT |
| | | CTACTCGCTGAGCAGCGTGGTGAGCGTGACCTCAAG |
| | | CAGCCAGCCCGTCACCTGCAACGTGGCCCACCCAGC |
| | | CACCAACACCAAAGTGGACAAGACCGTTGCGCCCTC |
| | | GACATGCAGCAAGCCCACGTGCCCACCCCCTGAACT |
| | | CCTGGGGGGACCGTCTGTCTTCATCTTCCCCCCAAAA |
| | | CCCAAGGACACCCTCATGATCTCACGCACCCCCGAG |
| | | GTCACATGCGTGGTGGTGGACGTGAGCCAGGATGAC |
| | | CCCGAGGTGCAGTTCACATGGTACATAAACAACGAG |
| | | CAGGTGCGCACCGCCCGGCCGCCGCTACGGGAGCAG |
| | | CAGTTCAACAGCACGATCCGCGTGGTCAGCACCCTC |
| | | CCCATCGCGCACCAGGACTGGCTGAGGGGCAAGGAG |
| | | TTCAAGTGCAAAGTCCACAACAAGGCACTCCCGGCC |
| | | CCCATCGAGAAAACCATCTCCAAAGCCAGAGGGCAG |
| | | CCCCTGGAGCCGAAGGTCTACACCATGGGCCCTCCC |
| | | CGGGAGGAGCTGAGCAGCAGGTCGGTCAGCCTGACC |
| | | TGCATGATCAACGGCTTCTACCCTTCCGACATCTCGG |
| | | TGGAGTGGGAGAAGAACGGGAAGGCAGAGGACAAC |
| | | TACAAGACCACGCCGGCCGTGCTGGACAGCGACGGC |
| | | TCCTACTTCCTCTACAGCAAGCTCTCAGTGCCCACGA |
| | | GTGAGTGGCAGCGGGGCGACGTCTTCACCTGCTCCG |
| | | TGATGCACGAGGCCTTGCACAACCACTACACGCAGA |
| | | AGTCCATCTCCCGCTCTCCGGGTAAATGA |
| | | (SEQ ID NO: 11) |
| | LC | ATGGACACGAGGGCACCCGCTCAGCTGCTGGGGCTC |
| | | CTGCTGCTCTGGCTCCCAGGTGCCACATTTGCCCAAG |
| | | TGCTGACCCAGACTCCATCCTCCGTGTCTGCAGCTGT |
| | | GGGAGGCACAGTCACCATCAATTGCCAGTCCGGTCA |
| | | GAGTGTTTATAATACCAACCAATTCTCCTGGATTCAG |
| | | CAGAAAGCAGGGCAGCCTCCCAAGTTCCTGATCGCC |
| | | GATGCATCCAAACTGGCATCTGGGGTCCCATCGCGG |
| | | TTCAGCGGCAGTGGATCTGGGACACACTTCACTCTC |
| | | ACCATCAGCGACGTCCAGTGTGACGATGCTGCCACT |
| | | TACTACTGTCTAGGCGGTTATGATTGTAGTAGTGCTG |
| | | ATTGCTCTGCTTTCGGCGGAGGGACCGAGGTGGTGG |
| | | TCGTTGGTGATCCAGTTGCACCTACTGTCCTCATCTT |
| | | CCCACCAGCTGCTGATCAGGTGGCAACTGGAACAGT |
| | | CACCATCGTGTGTGTGGCGAATAAATACTTTCCCGAT |
| | | GTCACCGTCACCTGGGAGGTGGATGGCACCACCCAA |
| | | ACAACTGGCATCGAGAACAGTAAAACACCGCAGAAT |
| | | TCTGCAGATTGTACCTACAACCTCAGCAGCACTCTGA |
| | | CACTGACCAGCACACAGTACAACAGCCACAAAGAGT |
| | | ACACCTGCAAGGTGACCCAGGGCACGACCTCAGTCG |
| | | TCCAGAGCTTCAATAGGGGTGACTGTTAG |
| | | (SEQ ID NO: 12) |

TABLE 6
Amino acid sequence of the Heavy Chain (HC) and Light Chain (LC)

| Antibody | | |
|---|---|---|
| Name | Chain | Amino Acid sequence |
| 21H1L4 | HC | METGLRWLLLVAVLKGVQCQSLEESGGDLVKPGASLT |
| | | LTCKASGFSFGSSAWIWWVRQAPGKGLEGVAWIYTGG |
| | | AGRLYYASWAKGRFTISKTSSTTVTLQMTSLTAADTAT |
| | | YFCARNSALWGPGTLVTVSSGQPKAPSVFPLAPCCGDT |
| | | PSSTVTLGCLVKGYLPEPVTVTWNSGTLTNGVRTFPSV |
| | | RQSSGLYSLSSVVSVTSSSQPVTCNVAHPATNTKVDKT |
| | | VAPSTCSKPTCPPPELLGGPSVFIFPPKPKDTLMISRTPE |
| | | VTCVVVDVSQDDPEVQFTWYINNEQVRTARPPLREQQ |
| | | FNSTIRVVSTLPIAHQDWLRGKEFKCKVHNKALPAPIE |
| | | KTISKARGQPLEPKVYTMGPPREELSSRSVSLTCMINGF |
| | | YPSDISVEWEKNGKAEDNYKTTPAVLDSDGSYFLYSKL |
| | | SVPTSEWQRGDVFTCSVMHEALHNHYTQKSISRSPGK |
| | | (SEQ ID NO: 13) |
| | LC | MDTRAPAQLLGLLLLWLPGATFAQVLTQTPSSVSAAV |
| | | GGTVTINCQSGQSVYNTNQFSWIQQKAGQPPKFLIADA |
| | | SKLASGVPSRFSGSGSGTHFTLTISDVQCDDAATYYCL |
| | | GGYDCSSADCSAFGGGTEVVVVGDPVAPTVLIFPPAAD |
| | | QVATGTVTIVCVANKYFPDVTVTWEVDGTTQTTGIEN |
| | | SKTPQNSADCTYNLSSTLTLTSTQYNSHKEYTCKVTQG |
| | | TTSVVQSFNRGDC |
| | | (SEQ ID NO: 14) |

### Example 2: Dot Blot Results In Vitro

Total RNA was isolated from cells using RNeasy mini kit (50) (74104, QIAGEN). mRNA was isolated from cells using Dynabeads™ mRNA Purification Kit (61006, QIAGEN). Synthesized oligonucleotides were commercially purchased. The RNA samples were serially diluted in RNase free water. 2 μL RNA was spotted on a nylon membrane Hybond N+ (Amersham RPN303B, GE), and crosslinked by UV light in SG Linker (Stratagene, 400071) for 125 mJ/cm2 at 254 nm, twice. The membrane was washed with TBST [20 mM Tris, pH 7.5, 150 mM NaCl, 0.1% (w/v) Tween® 20] at room temperature for 5 min, then blocked with AdvanBlock™-Chemi Blocking solution (R-03726-E10, Advansta) for 1 hr. The membrane was washed in TBST for 10 mins and then incubated in primary antibody 21H1L4 at 1 μg/ml with AdvanBlock™-Chemi Blocking solution overnight at 4° C. The membrane was washed three times in TBST for 10 min at room temperature, followed by an incubation with HRP-conjugated anti-rabbit antibody (ab97051, Abcam) at 1:20,000 dilution in AdvanBlock™-Chemi Blocking solution for 1 hr at room temperature. The membrane was washed four times in TBST for 10 min at room temperature, and developed with ECL (ab133406, Abcam, ECL Substrate Kit (High Sensitivity)). After imaging the immunoblot, the membrane was washed in TBST for 30 minutes. The membrane was then incubated with Streptavidin-AlexaFluor488 (1:5000 in TBST) and then imaged. After fluorescence imaging the blot is stained with methylene blue solution [0.2% (v/w) methylene blue, 0.4 M sodium acetate. 0.4 M acetic acid] for 30 min at room temperature. The stained membrane was washed briefly with H2O and imaged. The synthesized oligonucleotides tested included:

G-m6A:
(SEQ ID NO: 15)
5′Biotin-dB.dB.dB.dB.dB.dB.dB.dB.dB.dB.dG.(m6A).*dB.dB.dB.dB.dB.dB.dB.dB.dB.dB.-
Cy3-3′
A:
(SEQ ID NO: 16)
5′ Bi-dB.dB.dB.dB.dB.dB.dB.dB.dB.dB.d.A.*.dB.dB.dB.dB.dB.dB.dB.dB.dB.dB.-Cy3-3′
m6A:
(SEQ ID NO: 17)
5′ Bi-dB.dB.dB.dB.dB.dB.dB.dB.dB.dB.(m6A).*.dB.dB.dB.dB.dB.dB.dB.dB.dB.dB.-Cy3-3′
A-m6A:
(SEQ ID NO: 18)
5′ Bi-dB.dB.dB.dB.dB.dB.dB.dB.dB.dB.dA.(m6A).*.dB.dB.dB.dB.dB.dB.dB.dB.dB.dB.-
Cy3-3′
C-m6A:
(SEQ ID NO: 19)
5′ Bi-dB.dB.dB.dB.dB.dB.dB.dB.dB.dB.dC.(m6A).*.dB.dB.dB.dB.dB.dB.dB.dB.dB.dB.-
Cy3-3′
m1A:
(SEQ ID NO: 20)
5′Bi-dB.dB.dB.dB.dB.dB.dB.dB.dB.dB.m1A(ps)dB.dB.dB.dB.dB.dB.dB.dB.dB.dB.(AmC6)
(Cyanine3) 3′
m1G:
(SEQ ID NO: 21)
5′ Bi.dH.dH.dH.dH.dH.d.H.dH.dH.dH.dH.m1G.*.dH.dH.dH.d.H.d.H.d.H.d.H.d.H.d.H.d.H.
Cy3-3′ 3′
m22G:
(SEQ ID NO: 22)
5′ Bi.dH.dH.d.H.dH.dH.d.H.d.H.d.H.d.H.d.H.m2,2G.*.dH.dH.dH.d.H.d.H.d.H.d.H.dH.dH.
dH.Cy3-3′ 3′
m66A:
(SEQ ID NO: 23)
5′ Bi.dB.dB.dB.dB.dB.dB.dB.dB.dB.dB.(m66A).*.dB.dB.dB.dB.dB.dB.dB.dB.dB.dB.Cy3-
3′ 3′

The synthesized oligonucleotide terms are: d—2′-deoxyribonucleosides bases; m—2′-O methyl protection; B—equimolar mixture C/G/T; H— equimolar mixture C/A/T; *—phosphorothioate protection; (ps)—phosphorothioate protection; Bi—Biotin; Cy3—Cyanine3; and dB—dC/dG/dT equal molar mixture.

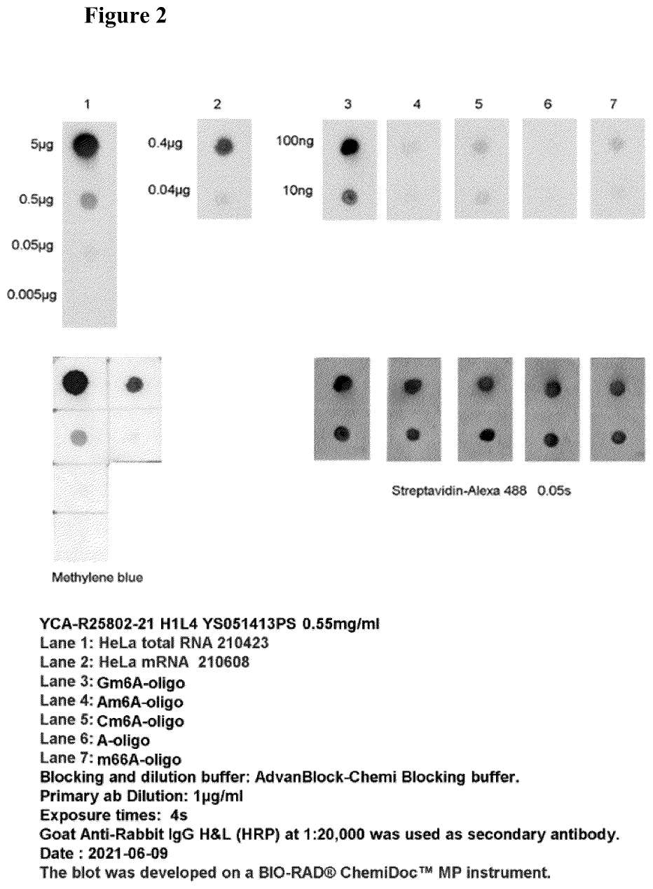

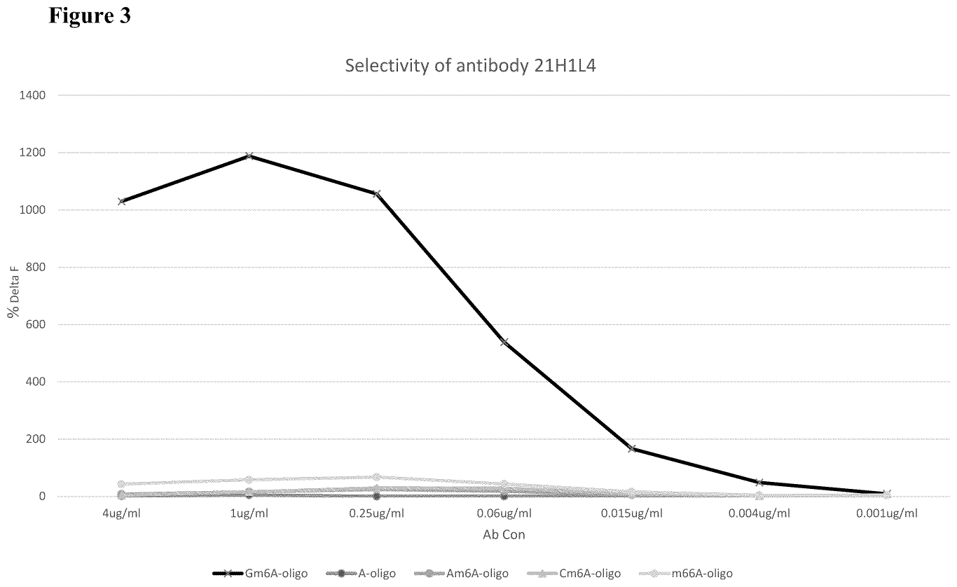

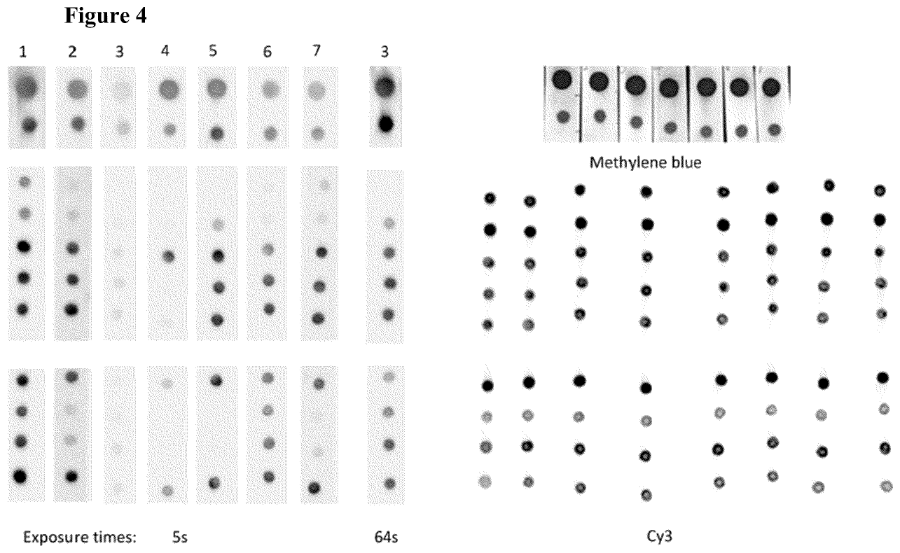

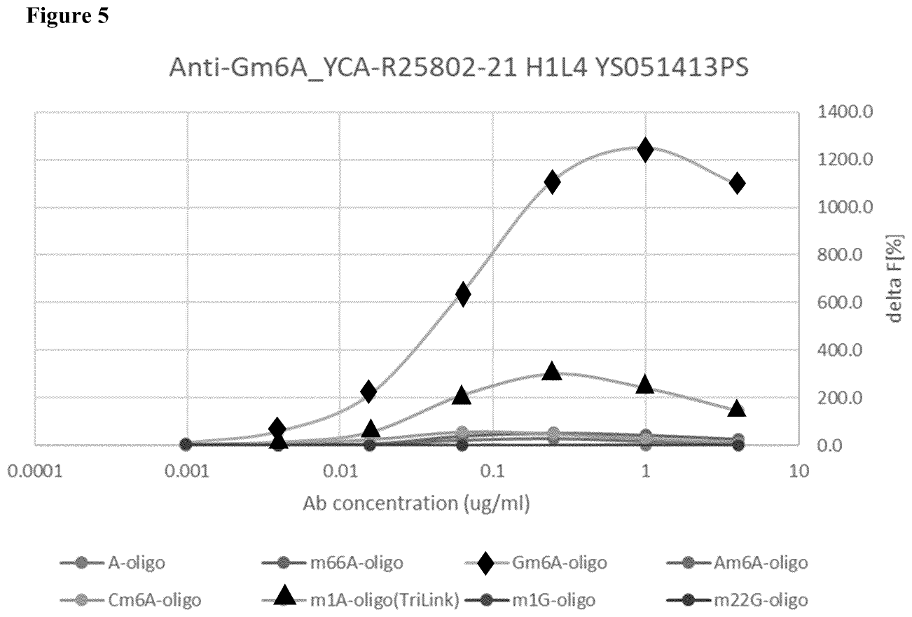

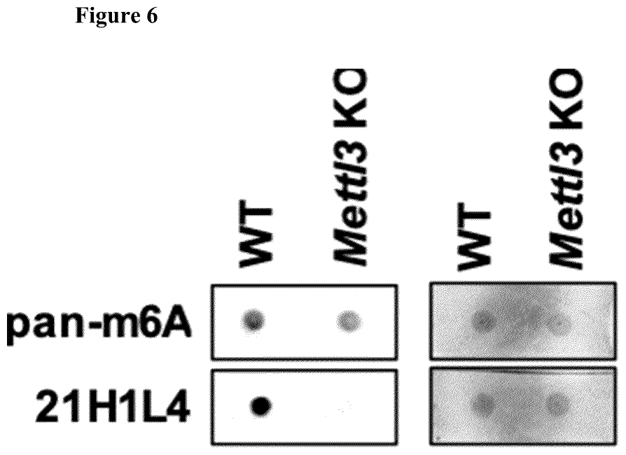

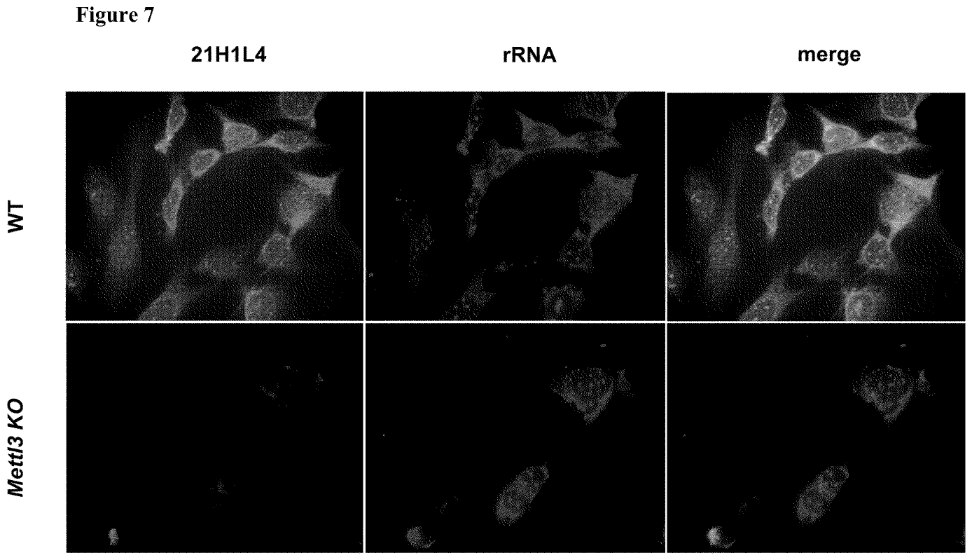

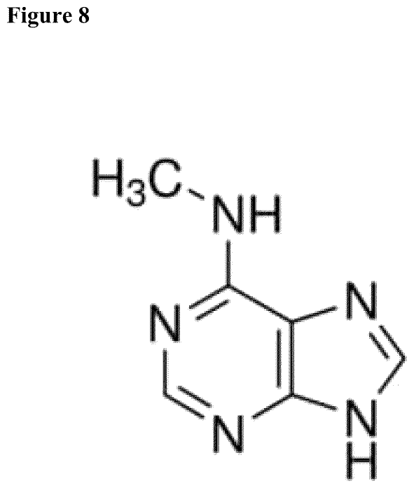

Results are shown in FIG. 2.

### Example 3: HTRF Results

5 ul of oligo was diluted in PBST to 100 nM and added to ProxiPlate-384 Plus plate followed by 5 ul of 1× Streptavidin-D2 (42 nM) (610SADLA, Cisbio) diluted in PBST, then 5 ul of Antibody 21H1L4 or purified IgG diluted in PBST to the desired concentrations (gradient concentration used 4 ug/ml, 1 ug/ml, 0.25 ug/ml, 0.06 ug/ml, 0.015 ug/ml, 0.004 ug/ml, 0.0001 ug/ml). Finally, 5 ul 1× Rabbit IgG-Tb (61PARTAB, Cisbio) (0.5 nM) diluted in PBST was added. This was sealed and incubated in the dark for 2 hours at room temperature. Plates were read using multimodal plate reader excitation at 340 nm and measure emission at 620 nm and 665 nm. The % Delta F was calculated using the following formula:

$$\begin{matrix}Δ F = \frac{{Ratio}_{sample} - {Ratio}_{background}}{{Ratio}_{background}} \% \\ \frac{Signal - Background}{Background}\end{matrix}$$

$$Ratio = acceptor emission ( 665 nm or 488 nm ) , donor emission ( 620 nm )$$

### Example 4: Dot Blot Results Ex Vivo

Mouse embryonic fibroblasts (MEFs) were isolated from Mettl3^{fl/fl} mouse and transformed by lentivirus-mediated induction of large T antigen (GenTarget Inc., #LVP016-Neo). The cells were further subjected to retrovirus-mediated induction of Cre-ERT2. After selection, the cells were treated with 500 nM 4-O-hydroxyltamoxifen (Santa Cruz Biotechnology, sc-3542) for 5 days to prepare Mettl3 knockout (KO) cells. Untreated cells were used as the wildtype (WT) control.

RNA dot blot was performed as previously described with some modifications (Meyer et al. 2012). Total RNA was isolated from cells using TRIzol reagent (ThermoFisher, 15596026). The total RNA pellet was dissolved in H2O at 2 μg/μL. 1 μL RNA was spotted on a nylon membrane (GE Healthcare, RPN303B), and crosslinked by UV light in Stratalinker® 1800 (Stratagene, 400071) for 250 mJ/cm2 at 254 nm, twice. The membrane was washed with TBST [20 mM Tris, pH 7.5, 150 mM NaCl, 0.1% (w/v) Tween® 20] at room temperature for 5 min, then blocked with 5% milk (Research Product International, M17200-1000.0) in TBST for 1 hr. The membrane was incubated in a primary antibody at 2 μg/mL in 5% milk/TBST for overnight at 4° C. The membrane was then washed three times in TBST for 5 min at room temperature, followed by an incubation with HRP-conjugated anti-rabbit antibody (VWR, 95017-556) at 1:5000 dilution in 5% milk/TBST for 1 hr at room temperature. The membrane was washed three times in TBST for 5 min at room temperature, and developed with ECL (VWR, 89168-782). After imaging the immunoblot, the membrane was stained with methylene blue solution [0.2% (v/w) methylene blue, 0.4 M sodium acetate. 0.4 M acetic acid] for 30 min at room temperature. The stained membrane was washed briefly with H₂O and imaged.

The primary antibodies used were either antibody 21H1L4 or a “pan-m6A antibody”, which is a rabbit polyclonal to N6-methyladenosine from Abcam, catalogue number 151230, at 1 ug/mL. Results are shown in FIG. 6, which shows that the pan-m6A antibody labels the WT and KO MEF RNA, whereas antibody 21H1L4 labels only WT MEF RNA.

### Example 5: Immunocytochemistry Results Ex Vivo

WT and KO MEFs were plated on glass bottom chamber slides coated with 0.1% gelatin (VWR, 62407-296; Fisher Scientific ES006B). 24 hr after seeding, cells were fixed with ice-cold methanol at −20° C. for 10 min. The cells were washed three times with PBS for 5 min at room temperature. After the last wash, PBS was replaced with ice-cold refolding buffer [1×PBS, 0.2% (w/v) Tween® 20, 10% formamide] and incubated on water bath at 37° C. for 10 min. The refolding step was repeated two more times. The cells were washed three times with PBS for 5 min at room temperature, followed by the second fixation in 4% formaldehyde (Life Technologies, 28906) for 10 min at room temperature. The cells were washed three times with PBS for 5 min at room temperature. After the last wash, the cells were treated with protease diluted at 1:4000 in PBS for 10 min at room temperature (ThermoFisher, 16742). After washing three times with PBS for 5 min at room temperature, the cells were treated with blocking buffer (5% serum) (Sigma Aldrich, D9663) in PBS supplemented with 0.2% (w/v) Tween® 20 and 20 U/mL SUPRase-IN (ThermoFisher, AM2694] for 1 hr at room temperature. The blocking was replaced with 10 μg/mL antibody 21H1L4 and 2.5 μg/mL anti-rRNA antibody (Santa Cruz Biotechnology, sc-33678) in blocking buffer for 4° C. overnight. After overnight staining with the primary antibody (21H1L4 or anti-rRNA antibody respectively), the cells were washed three times with PBS for 5 min at room temperature. The PBS was replaced with secondary antibody at 1:1000 dilution in blocking buffer for 1 hr at room temperature (ThermoFisher, A-21206; ThermoFisher, A10037). After washing three times with PBS for 5 min at room temperature, the cells were mounted with ProLong Diamond Antifade Mountant with DAPI (ThermoFisher, P36962) and sealed with a cover glass. Images were acquired using a Nikon TE-2000 inverted microscope with a 40× oil objective.

Results are shown in FIG. 7, which shows that 21H1L4 stains WT cells but not KO cells, whereas the rRNA antibody stains both WT and KO cells.

Noting the Examples in the present case and the common general knowledge, the skilled person would know how to generate further antibodies in a class that are selective for Guanine-N6-methyladenine (Gm6A) over Adenine-N6-methyladenine (Am6A). These further antibodies in a class may be raised against Carboxy C2 dG m6A (FIG. 1).

### REFERENCES

Bornaque, F., Delannoy, C. P., Courty, E., Rabhi, N., Carney, C., Rolland, L., Moreno, M., Gromada, X., Bourouh, C., Petit, P., et al. (2022). Glucose regulates m6a methylation of RNA in pancreatic islets. Cells 11.
Chang, M., Wang, Z., Gao, J., Yang, C., Feng, M., Niu, Y., Tong, W.-M., Bao, X., and Wang, R. (2022). METTL3-mediated RNA m6A Hypermethylation Promotes Tumorigenesis and GH Secretion of Pituitary Somatotroph Adenomas. J. Clin. Endocrinol. Metab. 107, 136-149.
Legrand, C., Tuorto, F., Hartmann, M., Liebers, R., Jacob, D., Helm, M., and Lyko, F. (2017). Statistically robust methylation calling for whole-transcriptome bisulfite sequencing reveals distinct methylation patterns for mouse RNAs. Genome Res. 27, 1589-1596.
Linder, B., Grozhik, A. V., Olarerin-George, A. O., Meydan, C., Mason, C. E., and Jaffrey, S. R. (2015). Single-nucleotide-resolution mapping of m6A and m6Am throughout the transcriptome. Nat. Methods 12, 767-772.
Maden, B. E. (1986). Identification of the locations of the methyl groups in 18 S ribosomal RNA from Xenopus laevis and man. J. Mol. Biol. 189, 681-699.
Maden, B. E. (1988). Locations of methyl groups in 28 S rRNA of Xenopus laevis and man. Clustering in the conserved core of molecule. J. Mol. Biol. 201, 289-314.
Ma, H., Wang, X., Cai, J., Dai, Q., Natchiar, S. K., Lv, R., Chen, K., Lu, Z., Chen, H., Shi, Y. G., et al. (2019). N6-Methyladenosine methyltransferase ZCCHC4 mediates ribosomal RNA methylation. Nat. Chem. Biol. 15, 88-94.
Patil, D. P., Pickering, B. F., and Jaffrey, S. R. (2018). Reading m6A in the Transcriptome: m6A-Binding Proteins. Trends Cell Biol. 28, 113-127.
Piekna-Przybylska, D., Decatur, W. A., and Fournier, M. J. (2008). The 3D rRNA modification maps database: with interactive tools for ribosome analysis. Nucleic Acids Res. 36, D178-83.
Shu, X., Cao, J., Cheng, M., Xiang, S., Gao, M., Li, T., Ying, X., Wang, F., Yue, Y., Lu, Z., et al. (2020). A metabolic labeling method detects m6A transcriptome-wide at single base resolution. Nat. Chem. Biol. 16, 887-895.
van Tran, N., Ernst, F. G. M., Hawley, B. R., Zorbas, C., Ulryck, N., Hackert, P., Bohnsack, K. E., Bohnsack, M. T., Jaffrey, S. R., Graille, M., et al. (2019). The human 18S rRNA m6A methyltransferase METTL5 is stabilized by TRMT112. Nucleic Acids Res. 47, 7719-7733.
Wang, Y., Xiao, Y., Dong, S., Yu, Q., and Jia, G. (2020). Antibody-free enzyme-assisted chemical approach for detection of N6-methyladenosine. Nat. Chem. Biol. 16, 896-903.
Wu, L., Wu, D., Ning, J., Liu, W., and Zhang, D. (2019). Changes of N6-methyladenosine modulators promote breast cancer progression. BMC Cancer 19, 326.
Yin, R., Chang, J., Li, Y., Gao, Z., Qiu, Q., Wang, Q., Han, G., Chai, J., Feng, M., Wang, P., et al. (2022). Differential m6A RNA landscapes across hematopoiesis reveal a role for IGF2BP2 in preserving hematopoietic stem cell function. Cell Stem Cell 29, 149-159.e7.
Jiang et al Signal Transduct Target Ther. 2021; 6: 74.

1. An antibody that is selective for Guanine-N6-methyladenine (Gm6A) over Adenine-N6-methyladenine (Am6A).

2. The antibody according to claim 1, wherein the antibody comprises a Heavy Chain Variable Region comprising:
(i) a variable heavy (VH) CDR1 that has the amino acid sequence of SEQ ID NO: 1;
(ii) a VH CDR2 that has the amino acid sequence of SEQ ID NO: 2; and
(iii) a VH CDR3 that has the amino acid sequence of SEQ ID NO: 3.

3. The antibody according to claim 2, wherein the antibody further comprises a Light Chain Variable Region comprising:
(iv) a variable light (VL) CDR1 that has the amino acid sequence of SEQ ID NO: 4;
(v) a VL CDR2 that has the amino acid sequence of SEQ ID NO: 5; and
(vi) a VL CDR3 that has the amino acid sequence of SEQ ID NO: 6.

4. An antibody according to claim 1 that can compete with an antibody comprising:
(a) a Heavy Chain Variable Region comprising:
(i) a variable heavy (VH) CDR1 that has the amino acid sequence of SEQ ID NO: 1;
(ii) a VH CDR2 that has the amino acid sequence of SEQ ID NO: 2; and
(iii) a VH CDR3 that has the amino acid sequence of SEQ ID NO: 3 and/or
(b) a Light Chain Variable Region comprising:
(iv) a variable light (VL) CDR1 that has the amino acid sequence of SEQ ID NO: 4;
(v) a VL CDR2 that has the amino acid sequence of SEQ ID NO: 5; and
(vi) a VL CDR3 that has the amino acid sequence of SEQ ID NO: 6,
for binding to Gm6A.

5. The antibody according to claim 4, wherein the antibody comprises:
(a) a Heavy Chain Variable Region comprising:
(i) a variable heavy (VH) CDR1 that has the amino acid sequence of SEQ ID NO: 1 or a sequence substantially homologous thereto;
(ii) a VH CDR2 that has the amino acid sequence of SEQ ID NO: 2 or a sequence substantially homologous thereto; and
(iii) a VH CDR3 that has the amino acid sequence of SEQ ID NO: 3 or a sequence substantially homologous thereto; and/or
(b) a Light Chain Variable Region comprising:
(iv) a variable light (VL) CDR1 that has the amino acid sequence of SEQ ID NO: 4 or a sequence substantially homologous thereto;
(v) a VL CDR2 that has the amino acid sequence of SEQ ID NO: 5 or a sequence substantially homologous thereto; and
(vi) a VL CDR3 that has the amino acid sequence of SEQ ID NO: 6 or a sequence substantially homologous thereto.

6. The antibody according to claim 5, wherein the substantially homologous sequence is a sequence containing exactly or no more than 5, 4, 3, 2 or 1 amino acid substitutions of the given CDR sequence.

7. The antibody according to claim 6, wherein each amino acid substitution is a conservative substitution.

8. The antibody according to claim 1, wherein the antibody has a Heavy Chain Variable Region having the sequence of SEQ ID NO: 7 or a sequence having at least 70%, 75%, 80%, 85%, 90%, 91%, 92%, 93%, 94%, 95%, 96%, 97%, 98% or at least 99% identity thereto; and/or a Light Chain Variable Region having the sequence of SEQ ID NO: 8 or a sequence having at least 70%, 75%, 80%, 85%, 90%, 91%, 92%, 93%, 94%, 95%, 96%, 97%, 98% or at least 99% identity thereto.

9. The antibody according to claim 1, wherein the antibody comprises a Heavy Chain Variable Region comprising the sequence of SEQ ID NO: 7 and a Light Chain Variable Region comprising the sequence of SEQ ID NO: 8.

10. The antibody according to claim 1, wherein the antibody is an IgA, IgD, IgE, IgM, or IgG antibody, or an antigen-binding fragment thereof such as a Fab, F(ab′)₂, Fv, scFv, diabody, monovalent IgG, scFv-Fc, minibody, or triabody.

11. A conjugate comprising the antibody of claim 1 conjugated to an agent, wherein the agent is preferably a selectable tag, a detectable label, a toxin, or a therapeutic agent.

12. A pharmaceutical composition comprising the antibody of claim 1 or a conjugate of the antibody, and a pharmaceutically acceptable carrier and/or excipient.

13. A nucleic acid molecule comprising a nucleotide sequence region encoding either:
a. the antibody of claim 1; or
b. a Heavy Chain Variable Region and/or a Light Chain Variable Region of the antibody.

14. An expression vector, or a set of expression vectors, comprising one or more nucleic acid molecules of claim 13, wherein the expression vector, or set of expression vectors, encodes and is suitable for expression of the antibody or a Heavy Chain Variable Region and/or a Light Chain Variable Region thereof.

15. A kit, optionally in one or more containers, comprising the:
(a) antibody according to claim 1; or
(b) a conjugate of the antibody;
and further comprising a:
(i) detectable label; and/or
(ii) agent for detecting the antibody or the conjugate.

16. A method of producing an antibody, comprising:
(a) culturing a host cell that comprises the expression vector or set of expression vectors according to claim 14, under conditions effective to express the encoded antibody regions; and
(b) obtaining the expressed antibody region.

17. Use of an antibody according to claim 1 or a conjugate of the antibody in an in vitro method of detecting, analysing or quantifying Gm6A levels in a sample, for example to assess the regulation of m6A modifications in mRNA.

18. A method of detecting, analysing and/or quantifying Gm6A, preferably Gm6A within an RNA molecule, comprising:
(a) contacting a sample with the antibody according to claim 1, or a conjugate the antibody, under conditions effective to allow the formation of complexes between Gm6A and the antibody; and
(b) detecting, analysing, and/or quantifying the complexes so formed.

19. A method of diagnosing a disease associated with aberrant Gm6A, comprising the steps of:
(a) contacting a subject or a test sample taken from the subject with an antibody according to claim 1 or a conjugate of the antibody, under conditions effective to allow the formation of complexes between the Gm6A and the antibody or conjugate; and
(b) measuring or detecting the presence, amount and/or location of antibody-antigen complex in the subject or test sample; and optionally
(c) comparing the presence, amount and/or location of antibody-antigen complex in the subject or test sample to a control.

20. A method for preventing, alleviating or treating a disease associated with aberrant Gm6A in a subject, comprising administering to a subject with the disease a therapeutically effective amount of the antibody according to claim 1, conjugate of the antibody, or pharmaceutical composition comprising the antibody or a conjugate of the antibody.
